(19) United States
(12) Patent Application Publication
(10) Pub. No.: US 2006/0263774 A1
(43) Pub. Date: Nov. 23, 2006

(54) Compositions and methods for the treatment of immune related diseases

(75) Inventors: Hilary Clark, South San Francisco, CA (US); Jill R Schoenfeld, Ashland, OR (US); Menno VanLookeren, San Francisco, CA (US); P Mickey Williams, Half Moon Bay, CA (US); William I Wood, Cupertino, CA (US); Thomas D Wu, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 10/533,519

(22) PCT Filed: Oct. 30, 2003
(86) PCT No.: PCT/US03/34312

§ 371 Date: Mar. 3, 2006

Related U.S. Application Data
(60) Provisional application No. 60/423,394, filed on Nov. 1, 2002.

(51) Int. Cl. C12Q 1/68; C07H 21/04; C12P 21/06; C07K 14/705; C07K 16/28

(52) U.S. Cl. 435/6; 435/69.1; 435/320.1; 435/325; 530/350; 530/388.22; 536/23.5

(57) ABSTRACT

The present invention relates to compositions containing novel proteins and methods of using those compositions for the diagnosis and treatment of immune related diseases.

### FIELD OF THE INVENTION

The present invention relates to compositions and methods useful for the diagnosis and treatment of immune related diseases.

### BACKGROUND OF THE INVENTION

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

Macrophages represent an ubiquitously distributed population of fixed and circulating mononuclear phagocytes that express a variety of functions including cytokine production, killing of microbes and tumor cells and processing and presentation of antigens. Macrophages originate in the bone marrow from stem cells that give rise to a bipotent granulocyte/macrophage cell population. Distinct granulocyte and macrophage colony forming cell lineages arise from GM-CSF under the influence of specific cytokines. Upon division, monoblasts give rise to promonocytes and monocytes in the bone marrow. From there, monocytes enter the circulation. In response to particular stimuli (e.g. infection or foreign bodies) monocytes migrate into tissues and organs where they differentiate into macrophages.

Macrophages in various tissues vary in their morphology and function and have been assigned different names, e.g. Kupffer cells in the liver, pulmonary and alveolar macrophages in the lung and microglial cells in the central nervous system. However, the relationship between blood monocytes and tissue macrophages remains unclear.

In the present study monocytes were differentiated into macrophages by adherence to plastic in the presence of a combination of human and bovine serum. After 7 days in culture, monocytes-derived macrophages display features typical of differentiated tissue macrophages including their ability to phagocytose opsonized particles, secretion of TNF-alpha upon lipopolysaccharide (LPS) stimulation, formation of processes and the presence of macrophage cell surface markers.

Using microarray technologies, gene transcripts from non-differentiated monocytes harvested before adhering were compared with those at 1 day and 7 days in culture. Genes selectively expressed in monocytes or macrophages could be used for the diagnosis and treatment of various chronic inflammatory or autoimmune diseases in the human. In particular, surface expressed molecules or transmembrane receptors involved in monocyte/macrophage adhesion and endothelial cell transmigration could provide novel targets to treat chronic inflammation by interference with the homing of these cells to the site of inflammation. In addition, transmembrane inhibitory receptors could be used to down-regulate monocyte/macrophage effector functions. Therapeutic molecules can be antibodies, peptides, fusion proteins or small molecules.

Despite the above research in monocyte/macrophages, there is a great need for additional diagnostic and therapeutic agents capable of detecting the presence of monocyte/macrophage mediated disorders in a mammal and for effectively reducing these disorders. Accordingly, it is an objective of the present invention to identify polypeptides that are differentially expressed in macrophages as compared to non-differentiated monocytes, and to use those polypeptides, and their encoding nucleic acids, to produce compositions of matter useful in the therapeutic treatment and diagnostic detection of monocyte/macrophage mediated disorders in mammals.

### SUMMARY OF THE INVENTION

A. Embodiments

The present invention concerns compositions and methods useful for the diagnosis and treatment of immune related disease in mammals, including humans. The present invention is based on the identification of proteins (including agonist and antagonist antibodies) which are a result of stimulation of the immune response in mammals. Immune related diseases can be treated by suppressing or enhancing the immune response. Molecules that enhance the immune response stimulate or potentiate the immune response to an antigen. Molecules which stimulate the immune response can be used therapeutically where enhancement of the immune response would be beneficial. Alternatively, molecules that suppress the immune response attenuate or reduce the immune response to an antigen (e.g., neutralizing antibodies) can be used therapeutically where attenuation of the immune response would be beneficial (e.g., inflammation). Accordingly, the PRO polypeptides, agonists and antagonists thereof are also useful to prepare medicines and medicaments for the treatment of immune-related and inflammatory diseases. In a specific aspect, such medicines and medicaments comprise a therapeutically effective amount of a PRO polypeptide, agonist or antagonist thereof with a pharmaceutically acceptable carrier. Preferably, the admixture is sterile.

In a further embodiment, the invention concerns a method of identifying agonists or antagonists to a PRO polypeptide which comprises contacting the PRO polypeptide with a candidate molecule and monitoring a biological activity mediated by said PRO polypeptide. Preferably, the PRO polypeptide is a native sequence PRO polypeptide. In a specific aspect, the PRO agonist or antagonist is an anti-PRO antibody.

In another embodiment, the invention concerns a composition of matter comprising a PRO polypeptide or an agonist or antagonist antibody which binds the polypeptide in admixture with a carrier or excipient. In one aspect, the composition comprises a therapeutically effective amount of the polypeptide or antibody. In another aspect, when the composition comprises an immune stimulating molecule, the composition is useful for: (a) increasing infiltration of inflammatory cells into a tissue of a mammal in need thereof, (b) stimulating or enhancing an immune response in a mammal in need thereof (c) increasing the proliferation of monocytes/macrophages in a mammal in need thereof in response to an antigen, (d) stimulating the activity of monocytes/macrophages or (e) increasing the vascular permeability. In a further aspect, when the composition comprises an immune inhibiting molecule, the composition is useful for: (a) decreasing infiltration of inflammatory cells into a tissue of a mammal in need thereof, (b) inhibiting or reducing an immune response in a mammal in need thereof, (c) decreasing the activity of monocytes/macrophages or (d) decreasing the proliferation of monocytes/macrophages in a mammal in need thereof in response to an antigen. In another aspect, the composition comprises a further active ingredient, which may, for example, be a further antibody or a cytotoxic or chemotherapeutic agent. Preferably, the composition is sterile.

In another embodiment, the invention concerns a method of treating an immune related disorder in a mammal in need thereof comprising administering to the mammal an effective amount of a PRO polypeptide, an agonist thereof, or an antagonist thereto. In a preferred aspect, the immune related disorder is selected from the group consisting of systemic lupus erythematosis, rheumatoid arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In another embodiment, the invention provides an antibody which specifically binds to any of the above or below described polypeptides. Optionally, the antibody is a monoclonal antibody, humanized antibody, antibody fragment or single-chain antibody. In one aspect, the present invention concerns an isolated antibody which binds a PRO polypeptide. In another aspect, the antibody mimics the activity of a PRO polypeptide (an agonist antibody) or conversely the antibody inhibits or neutralizes the activity of a PRO polypeptide (an antagonist antibody). In another aspect, the antibody is a monoclonal antibody, which preferably has nonhuman complementarity determining region (CDR) residues and human framework region (FR) residues. The antibody may be labeled and may be immobilized on a solid support. In a further aspect, the antibody is an antibody fragment, a monoclonal antibody, a single-chain antibody, or an anti-idiotypic antibody.

In yet another embodiment, the present invention provides a composition comprising an anti-PRO antibody in admixture with a pharmaceutically acceptable carrier. In one aspect, the composition comprises a therapeutically effective amount of the antibody. Preferably, the composition is sterile. The composition may be administered in the form of a liquid pharmaceutical formulation, which may be preserved to achieve extended storage stability. Alternatively, the antibody is a monoclonal antibody, an antibody fragment, a humanized antibody, or a single-chain antibody.

In a further embodiment, the invention concerns an article of manufacture, comprising:

(a) a composition of matter comprising a PRO polypeptide or agonist or antagonist thereof;

(b) a container containing said composition; and

(c) a label affixed to said container, or a package insert included in said container referring to the use of said PRO polypeptide or agonist or antagonist thereof in the treatment of an immune related disease. The composition may comprise a therapeutically effective amount of the PRO polypeptide or the agonist or antagonist thereof.

In yet another embodiment, the present invention concerns a method of diagnosing an immune related disease in a mammal, comprising detecting the level of expression of a gene encoding a PRO polypeptide (a) in a test sample of tissue cells obtained from the mammal, and (b) in a control sample of known normal tissue cells of the same cell type, wherein a higher or lower expression level in the test sample as compared to the control sample indicates the presence of immune related disease in the mammal from which the test tissue cells were obtained.

In another embodiment, the present invention concerns a method of diagnosing an immune disease in a mammal, comprising (a) contacting an anti-PRO antibody with a test sample of tissue cells obtained from the mammal, and (b) detecting the formation of a complex between the antibody and a PRO polypeptide, in the test sample; wherein the formation of said complex is indicative of the presence or absence of said disease. The detection may be qualitative or quantitative, and may be performed in comparison with monitoring the complex formation in a control sample of known normal tissue cells of the same cell type. A larger quantity of complexes formed in the test sample indicates the presence or absence of an immune disease in the mammal from which the test tissue cells were obtained. The antibody preferably carries a detectable label. Complex formation can be monitored, for example, by light microscopy, flow cytometry, fluorimetry, or other techniques known in the art. The test sample is usually obtained from an individual suspected of having a deficiency or abnormality of the immune system.

In another embodiment, the invention provides a method for determining the presence of a PRO polypeptide in a sample comprising exposing a test sample of cells suspected of containing the PRO polypeptide to an anti-PRO antibody and determining the binding of said antibody to said cell sample. In a specific aspect, the sample comprises a cell suspected of containing the PRO polypeptide and the antibody binds to the cell. The antibody is preferably detectably labeled and/or bound to a solid support.

In another embodiment, the present invention concerns an immune-related disease diagnostic kit, comprising an anti-PRO antibody and a carrier in suitable packaging. The kit preferably contains instruction for using the antibody to detect the presence of the PRO polypeptide. Preferably the carrier is pharmaceutically acceptable.

In another embodiment, the present invention concerns a diagnostic kit, containing an anti-PRO antibody in suitable packaging. The kit preferably contains instructions for using the antibody to detect the PRO polypeptide.

In another embodiment, the invention provides a method of diagnosing an immune-related disease in a mammal which comprises detecting the presence or absence or a PRO polypeptide in a test sample of tissue cells obtained from said mammal, wherein the presence or absence of the PRO polypeptide in said test sample is indicative of the presence of an immune-related disease in said mammal.

In another embodiment, the present invention concerns a method for identifying an agonist of a PRO polypeptide comprising:

(a) contacting cells and a test compound to be screened under conditions suitable for the induction of a cellular response normally induced by a PRO polypeptide; and

(b) determining the induction of said cellular response to determine if the test compound is an effective agonist, wherein the induction of said cellular response is indicative of said test compound being an effective agonist.

In another embodiment, the invention concerns a method for identifying a compound capable of inhibiting the activity of a PRO polypeptide comprising contacting a candidate compound with a PRO polypeptide under conditions and for a time sufficient to allow these two components to interact and determining whether the activity of the PRO polypeptide is inhibited. In a specific aspect, either the candidate compound or the PRO polypeptide is immobilized on a solid support. In another aspect, the non-immobilized component carries a detectable label. In a preferred aspect, this method comprises the steps of

(a) contacting cells and a test compound to be screened in the presence of a PRO polypeptide under conditions suitable for the induction of a cellular response normally induced by a PRO polypeptide; and

(b) determining the induction of said cellular response to determine if the test compound is an effective antagonist.

In another embodiment, the invention provides a method for identifying a compound that inhibits the expression of a PRO polypeptide in cells that normally express the polypeptide, wherein the method comprises contacting the cells with a test compound and determining whether the expression of the PRO polypeptide is inhibited. In a preferred aspect this method comprises the steps of:

(a) contacting cells and a test compound to be screened under conditions suitable for allowing expression of the PRO polypeptide; and

(b) determining the inhibition of expression of said polypeptide.

In yet another embodiment, the present invention concerns a method for treating an immune-related disorder in a mammal that suffers therefrom comprising administering to the mammal a nucleic acid molecule that codes for either (a) a PRO polypeptide, (b) an agonist of a PRO polypeptide or (c) an antagonist of a PRO polypeptide, wherein said agonist or antagonist may be an anti-PRO antibody. In a preferred embodiment, the mammal is human. In another preferred embodiment, the nucleic acid is administered via ex vivo gene therapy. In a further preferred embodiment, the nucleic acid is comprised within a vector, more preferably an adenoviral, adeno-associated viral, lentiviral or retroviral vector.

In yet another aspect, the invention provides a recombinant viral particle comprising a viral vector consisting essentially of a promoter, nucleic acid encoding (a) a PRO polypeptide, (b) an agonist polypeptide of a PRO polypeptide, or (c) an antagonist polypeptide of a PRO polypeptide, and a signal sequence for cellular secretion of the polypeptide, wherein the viral vector is in association with viral structural proteins. Preferably, the signal sequence is from a mammal, such as from a native PRO polypeptide.

In a still further embodiment, the invention concerns an ex vivo producer cell comprising a nucleic acid construct that expresses retroviral structural proteins and also comprises a retroviral vector consisting essentially of a promoter, nucleic acid encoding (a) a PRO polypeptide, (b) an agonist polypeptide of a PRO polypeptide or (c) an antagonist polypeptide of a PRO polypeptide, and a signal sequence for cellular secretion of the polypeptide, wherein said producer cell packages the retroviral vector in association with the structural proteins to produce recombinant retroviral particles.

In a still further embodiment, the invention provides a method of increasing the activity of monocytes/macrophages in a mammal comprising administering to said mammal (a) a PRO polypeptide, (b) an agonist of a PRO polypeptide, or (c) an antagonist of a PRO polypeptide, wherein the activity of monocytes/macrophages in the mammal is increased.

In a still further embodiment, the invention provides a method of decreasing the activity of monocytes/macrophages in a mammal comprising administering to said mammal (a) a PRO polypeptide, (b) an agonist of a PRO polypeptide, or (c) an antagonist of a PRO polypeptide, wherein the activity of monocytes/macrophages in the mammal is decreased.

In a still further embodiment, the invention provides a method of increasing the proliferation of monocytes/macrophages in a mammal comprising administering to said mammal (a) a PRO polypeptide, (b) an agonist of a PRO polypeptide, or (c) an antagonist of a PRO polypeptide, wherein the proliferation of monocytes/macrophages in the mammal is increased.

In a still further embodiment, the invention provides a method of decreasing the proliferation of monocytes/macrophages in a mammal comprising administering to said mammal (a) a PRO polypeptide, (b) an agonist of a PRO polypeptide, or (c) an antagonist of a PRO polypeptide, wherein the proliferation of monocytes/macrophages in the mammal is decreased.

B. Additional Embodiments

In other embodiments of the present invention, the invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which specifically binds to any of the above or below described polypeptides. Optionally, the antibody is a monoclonal antibody, humanized antibody, antibody fragment or single-chain antibody.

In yet other embodiments, the invention provides oligonucleotide probes useful for isolating genomic and cDNA nucleotide sequences or as antisense probes, wherein those probes may be derived from any of the above or below described nucleotide sequences.

In other embodiments, the invention provides an isolated nucleic acid molecule comprising a nucleotide sequence that encodes a PRO polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO polypeptide having a full-length amino acid sequence as disclosed herein, an amino acid sequence lacking the signal peptide as disclosed herein, an extracellular domain of a membrane protein, with or without the signal peptide, as disclosed herein or any other specifically defined fragment of the full-length amino acid sequence as disclosed herein, or (a) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO polypeptide cDNA as disclosed herein, the coding sequence of a PRO polypeptide lacking the signal peptide as disclosed herein, the coding sequence of an extracellular domain of a transmembrane PRO polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs as disclosed herein, or (b) the complement of the DNA molecule of (a).

Another aspect the invention provides an isolated nucleic acid molecule comprising a nucleotide sequence encoding a PRO polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO polypeptides are contemplated.

Another embodiment is directed to fragments of a PRO polypeptide coding sequence, or the complement thereof, that may find use as, for example, hybridization probes, for encoding fragments of a PRO polypeptide that may optionally encode a polypeptide comprising a binding site for an anti-PRO antibody or as antisense oligonucleotide probes. Such nucleic acid fragments are usually at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. It is noted that novel fragments of a PRO polypeptide-encoding nucleotide sequence may be determined in a routine manner by aligning the PRO polypeptide-encoding nucleotide sequence with other known nucleotide sequences using any of a number of well known sequence alignment programs and determining which PRO polypeptide-encoding nucleotide sequence fragment(s) are novel. All of such PRO polypeptide-encoding nucleotide sequences are contemplated herein. Also contemplated are the PRO polypeptide fragments encoded by these nucleotide molecule fragments, preferably those PRO polypeptide fragments that comprise a binding site for an anti-PRO antibody.

In another embodiment, the invention provides isolated PRO polypeptide encoded by any of the isolated nucleic acid sequences herein above identified.

In a certain aspect, the invention concerns an isolated PRO polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs as disclosed herein.

In a specific aspect, the invention provides an isolated PRO polypeptide without the N-terminal signal sequence and/or the initiating methionine and is encoded by a nucleotide sequence that encodes such an amino acid sequence as herein before described. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

Another aspect the invention provides an isolated PRO polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

In yet another embodiment, the invention concerns agonists and antagonists of a native PRO polypeptide as defined herein. In a particular embodiment, the agonist or antagonist is an anti-PRO antibody or a small molecule.

In a further embodiment, the invention concerns a method of identifying agonists or antagonists to a PRO polypeptide which comprise contacting the PRO polypeptide with a candidate molecule and monitoring a biological activity mediated by said PRO polypeptide. Preferably, the PRO polypeptide is a native PRO polypeptide.

In a still further embodiment, the invention concerns a composition of matter comprising a PRO polypeptide, or an agonist or antagonist of a PRO polypeptide as herein described, or an anti-PRO antibody, in combination with a carrier. Optionally, the carrier is a pharmaceutically acceptable carrier.

Another embodiment of the present invention is directed to the use of a PRO polypeptide, or an agonist or antagonist thereof as herein before described, or an anti-PRO antibody, for the preparation of a medicament useful in the treatment of a condition which is responsive to the PRO polypeptide, an agonist or antagonist thereof or an anti-PRO antibody.

### BRIEF DESCRIPTION OF THE DRAWINGS

In the list of figures for the present application, specific cDNA sequences which are differentially expressed in differentiated macrophages as compared to normal undifferentiated monocytes are individually identified with a specific alphanumerical designation. These cDNA sequences are differentially expressed in monocytes that are specifically treated as described in Example 1 below. If start and/or stop codons have been identified in a cDNA sequence shown in the attached figures, they are shown in bold and underlined font, and the encoded polypeptide is shown in the next consecutive figure.

The FIGS. 1-2517 show the nucleic acids of the invention and their encoded PRO polypeptides. Also included, for convenience is a List of Figures attached hereto as Appendix A, which gives the figure number and the corresponding DNA or PRO number.

List of Figures

FIG. 1: DNA227321, NP_001335.1, 200046_at
FIG. 2: PRO37784
FIG. 3: DNA304680, HSPCB, 200064_at
FIG. 4: PRO71106
FIG. 5: DNA328347, NP_002146.1, 117_at
FIG. 6: PRO58142
FIG. 7A-B: DNA328348, MAP4, 243_g_at
FIG. 8: PRO84209
FIG. 9: DNA83128, NP_002979.1, 32128_at
FIG. 10: PRO2601
FIG. 11: DNA272223, NP_004444.1, 33494_at
FIG. 12: PRO60485
FIG. 13: DNA327522, NP_000396.1, 33646_g_at
FIG. 14: PRO2874
FIG. 15: DNA328349, NP_004556.1, 33760_at
FIG. 16: PRO84210
FIG. 17A-B: DNA328350, NP_056155.1, 34764_at
FIG. 18: PRO84211
FIG. 19: DNA328351, NP_006143.1, 35974_at
FIG. 20: PRO84212
FIG. 21: DNA328352, NP_004183.1, 36553_at
FIG. 22: PRO84213
FIG. 23: DNA271996, NP_004928.1, 36566_at
FIG. 24: PRO60271
FIG. 25: DNA326969, NP_036455.1, 36711_at
FIG. 26: PRO83282
FIG. 27: DNA304703, NP_005923.1, 36830_at
FIG. 28: PRO71129
FIG. 29: DNA328353, AAB72234.1, 37079_at
FIG. 30: PRO84214
FIG. 31: DNA103289, NP_006229.1, 37152_at
FIG. 32: PRO4619
FIG. 33A-B: DNA255096, NP_055449.1, 37384_at
FIG. 34: PRO50180
FIG. 35: DNA256295, NP_002310.1, 37796_at
FIG. 36: PRO51339
FIG. 37: DNA328354, PARVB, 37965_at
FIG. 38: PRO84215
FIG. 39: DNA53531, NP_001936.1, 38037_at
FIG. 40: PRO131
FIG. 41: DNA254127, NP_008925.1, 38241_at
FIG. 42: PRO49242
FIG. 43: DNA328355, NP_006471.2, 38290_at
FIG. 44: PRO84216
FIG. 45: DNA328356, BC013566, 39248_at
FIG. 46: PRO38028
FIG. 47: DNA328357, 1452321.2, 39582_at
FIG. 48: PRO84217
FIG. 49A-B: DNA328358, STK10, 40420_at
FIG. 50: PRO84218
FIG. 51A-B: DNA328359, BAA21572.1, 41386_i_at
FIG. 52: PRO84219
FIG. 53A-D: DNA328360, NP_055061.1, 41660_at
FIG. 54: PRO84220
FIG. 55: DNA327526, BC001698, 45288_at
FIG. 56: PRO83574
FIG. 57A-B: DNA328361, BAA92570.1, 47773_at
FIG. 58: PRO84221
FIG. 59: DNA328362, NP_060312.1, 48106_at
FIG. 60: PRO84222
FIG. 61: DNA328363, DNA328363, 52651_at
FIG. 62: PRO84685
FIG. 63: DNA328364, NP_068577.1, 52940_at
FIG. 64: PRO84223
FIG. 65A-B: DNA327528, BAB33338.1, 55081_at
FIG. 66: PRO83576
FIG. 67: DNA225650, NP_057246.1, 48825_at
FIG. 68: PRO36113
FIG. 69: DNA328365, NP_060541.1, 58780_s_at
FIG. 70: PRO84224
FIG. 71: DNA328366, NP_079233.1, 59375_at
FIG. 72: PRO84225
FIG. 73: DNA328367, NP_079108.2, 60471_at
FIG. 74: PRO84226
FIG. 75: DNA327876, NP_005081.1, 60528_at
FIG. 76: PRO83815
FIG. 77A-B: DNA328368, 1503444.3, 87100_at
FIG. 78: PRO84227
FIG. 79: DNA328369, BC007634, 90610_at
FIG. 80A-B: DNA328370, NP_001273.1,
200615_s_at
FIG. 81: PRO84228
FIG. 82: DNA323806, NP_075385.1, 200644_at
FIG. 83: PRO80555
FIG. 84: DNA327532, GLUL, 200648_s_at
FIG. 85: PRO71134
FIG. 86: DNA227055, NP_002625.1, 200658_s_at
FIG. 87: PRO37518
FIG. 88: DNA325702, NP_001771.1, 200663_at
FIG. 89: PRO283
FIG. 90: DNA83172, NP_003109.1, 200665_s_at
FIG. 91: PRO2120
FIG. 92: DNA328371, NP_004347.1, 200675_at
FIG. 93: PRO4866
FIG. 94A-B: DNA328372, 105551.7, 200685_at
FIG. 95: PRO84229
FIG. 96: DNA324633, BC000478, 200691_s_at
FIG. 97: PRO81277
FIG. 98: DNA324633, NP_004125.2, 200692_s_at
FIG. 99: PRO81277
FIG. 100: DNA88350, NP_000168.1, 200696_s_at
FIG. 101: PRO2758
FIG. 102: DNA328373, AB034747, 200704_at
FIG. 103: PRO84230
FIG. 104: DNA328374, NP_004853.1, 200706_s_at
FIG. 105: PRO84231
FIG. 106: DNA328375, NP_002071.1, 200708_at
FIG. 107: PRO80880
FIG. 108: DNA328376, NP_001210.1, 200755_s_at
FIG. 109: PRO1015
FIG. 110A-B: DNA269826, NP_003195.1,
200758_s_at
FIG. 111: PRO58228
FIG. 112: DNA325414, NP_001900.1, 200766_at
FIG. 113: PRO292
FIG. 114A-C: DNA188738, NP_002284.2, 200771_at
FIG. 115: PRO25580
FIG. 116: DNA328377, NP_003759.1, 200787_s_at
FIG. 117: PRO84232
FIG. 118: DNA270954, NP_001089.1, 200793_s_at
FIG. 119: PRO59285
FIG. 120: DNA272928, NP_055579.1, 200794_x_at
FIG. 121: PRO61012
FIG. 122A-B: DNA327536, BC017197, 200797_s_at
FIG. 123: PRO37003
FIG. 124: DNA287211, NP_002147.1, 200806_s_at
FIG. 125: PRO69492
FIG. 126: DNA326655, NP_002803.1, 200820_at
FIG. 127: PRO83005
FIG. 128A-B: DNA328378, AB032261, 200832_s_at
FIG. 129: PRO84233
FIG. 130: DNA103558, NP_005736.1, 200837_at
FIG. 131: PRO4885
FIG. 132: DNA196817, NP_001899.1, 200838_at
FIG. 133: PRO3344
FIG. 134A-B: DNA327537, NP_004437.1,
200842_s_at
FIG. 135: PRO83581
FIG. 136: DNA323982, NP_004896.1, 200844_s_at
FIG. 137: PRO80709
FIG. 138: DNA323876, NP_006612.2, 200850_s_at
FIG. 139: PRO80619
FIG. 140A-B: DNA228029, NP_055577.1, 200862_at
FIG. 141: PRO38492
FIG. 142: DNA328379, BC015869, 200878_at
FIG. 143: PRO84234
FIG. 144: DNA325584, NP_002005.1, 200895_s_at
FIG. 145: PRO59262
FIG. 146A-B: DNA274281, NP_036347.1,
200899_s_at
FIG. 147: PRO62204
FIG. 148: DNA226028, NP_002346.1, 200900_s_at
FIG. 149: PRO36491
FIG. 150: DNA326819, NP_000678.1, 200903_s_at
FIG. 151: PRO83152
FIG. 152: DNA328380, HSHLAEHCM, 200904_at
FIG. 153: DNA328381, NP_005507.1, 200905_x_at
FIG. 154: PRO84236
FIG. 155: DNA272695, NP_001722.1, 200920_s_at
FIG. 156: PRO60817
FIG. 157: DNA327255, NP_002385.2, 200924_s_at
FIG. 158: PRO57298
FIG. 159: DNA327540, NP_006818.1, 200929_at
FIG. 160: PRO38005
FIG. 161: DNA225878, NP_004334.1, 200935_at
FIG. 162: PRO36341
FIG. 163: DNA328382, 160963.2, 200941_at
FIG. 164: PRO84237
FIG. 165: DNA328383, NP_004956.3, 200944_s_at
FIG. 166: PRO84238
FIG. 167A-B: DNA287217, NP_001750.1,
200953_s_at
FIG. 168: PRO36766
FIG. 169: DNA328384, NP_036380.2, 200961_at
FIG. 170: PRO84239
FIG. 171: DNA328385, AK001310, 200972_at
FIG. 172: PRO730
FIG. 173: DNA326040, NP_005715.1, 200973_s_at
FIG. 174: PRO730
FIG. 175: DNA324110, NP_005908.1, 200978_at
FIG. 176: PRO4918
FIG. 177: DNA328386, NP_000602.1, 200983_x_at
FIG. 178: PRO2697
FIG. 179: DNA275408, NP_001596.1, 201000_at
FIG. 180: PRO63068
FIG. 181: DNA328387, NP_001760.1, 201005_at
FIG. 182: PRO4769
FIG. 183: DNA103593, NP_000174.1, 201007_at
FIG. 184: PRO4917
FIG. 185: DNA304713, NP_006463.2, 201008_s_at
FIG. 186: PRO71139
FIG. 187: DNA328388, BC010273, 201013_s_at
FIG. 188: PRO84240
FIG. 189: DNA328389, NP_006861.1, 201021_s_at
FIG. 190: PRO84241
FIG. 191: DNA328390, NP_002291.1, 201030_x_at
FIG. 192: PRO82116
FIG. 193: DNA196628, NP_005318.1, 201036_s_at
FIG. 194: PRO25105
FIG. 195: DNA287372, NP_002618.1, 201037_at
FIG. 196: PRO69632
FIG. 197: DNA328391, NP_004408.1, 201041_s_at
FIG. 198: PRO84242
FIG. 199: DNA196484, DNA196484, 201042_at
FIG. 200: DNA227143, NP_036400.1, 201050_at
FIG. 201: PRO37606
FIG. 202: DNA328392, 1500938.11, 201051_at
FIG. 203: PRO84243
FIG. 204: DNA328261, AF130103, 201060_x_at
FIG. 205: DNA325001, NP_002794.1, 201068_s_at
FIG. 206: PRO81592
FIG. 207: DNA328393, NP_001651.1, 201096_s_at
FIG. 208: PRO81010
FIG. 209: DNA328394, AF131738, 201103_x_at
FIG. 210A-B: DNA328395, NP_056198.1,
201104_x_at
FIG. 211: PRO84245
FIG. 212: DNA328396, NP_002076.1, 201106_at
FIG. 213: PRO84246
FIG. 214: DNA328397, NP_002622.1, 201118_at
FIG. 215: PRO84247
FIG. 216: DNA328398, NP_002204.1, 201125_s_at
FIG. 217: PRO34737
FIG. 218: DNA325398, NP_004083.2, 201135_at
FIG. 219: PRO81930
FIG. 220: DNA88520, NP_002501.1, 201141_at
FIG. 221: PRO2824
FIG. 222: DNA324480, NP_001544.1, 201163_s_at
FIG. 223: PRO81141
FIG. 224: DNA151802, NP_003661.1, 201169_s_at
FIG. 225: PRO12890
FIG. 226: DNA226662, NP_057043.1, 201175_at
FIG. 227: PRO37125
FIG. 228: DNA88066, NP_002328.1, 201186_at
FIG. 229: PRO2638
FIG. 230: DNA273342, NP_005887.1, 201193_at
FIG. 231: PRO61345
FIG. 232: DNA328399, NP_003000.1, 201194_at
FIG. 233: PRO84248
FIG. 234A-B: DNA103453, HUME16GEN,
201195_s_at
FIG. 235: PRO4780
FIG. 236: DNA328400, NP_003842.1, 201200_at
FIG. 237: PRO1409
FIG. 238: DNA327542, NP_000091.1, 201201_at
FIG. 239: PRO83582
FIG. 240: DNA103488, NP_002583.1, 201202_at
FIG. 241: PRO4815
FIG. 242: DNA328401, BC013678, 201212_at
FIG. 243A-B: DNA328402, NP_073713.1,
201220_x_at
FIG. 244: PRO84249
FIG. 245: DNA325380, NP_004995.1, 201226_at
FIG. 246: PRO81914
FIG. 247: DNA226615, NP_001668.1, 201242_s_at
FIG. 248: PRO37078
FIG. 249: DNA328403, NP_037462.1, 201243_s_at
FIG. 250: PRO84250
FIG. 251: DNA270950, NP_003182.1, 201263_at
FIG. 252: PRO59281
FIG. 253A-B: DNA328404, NP_003321.1, 201266_at
FIG. 254: PRO84251
FIG. 255: DNA97290, NP_002503.1, 201268_at
FIG. 256: PRO3637
FIG. 257: DNA325028, NP_001619.1, 201272_at
FIG. 258: PRO81617
FIG. 259: DNA328405, NP_112556.1, 201277_s_at
FIG. 260: PRO84252
FIG. 261: DNA328406, NP_001334.1, 201279_s_at
FIG. 262: PRO84253
FIG. 263: DNA328407, WSB1, 201296_s_at
FIG. 264: PRO84254
FIG. 265: DNA328408, NP_060713.1, 201308_s_at
FIG. 266: PRO84255
FIG. 267: DNA325595, NP_001966.1, 201313_at
FIG. 268: PRO38010
FIG. 269: DNA255078, NP_006426.1, 201315_x_at
FIG. 270: PRO50165
FIG. 271: DNA150781, NP_001414.1, 201324_at
FIG. 272: PRO12467
FIG. 273: DNA328409, NP_002075.2, 201348_at
FIG. 274: PRO81281
FIG. 275: DNA324475, NP_004172.2, 201387_s_at
FIG. 276: PRO81137
FIG. 277: DNA226353, NP_005769.1, 201395_at
FIG. 278: PRO36816
FIG. 279: DNA328410, NP_004519.1, 201403_s_at
FIG. 280: PRO60174
FIG. 281A-B: DNA328411, 1400253.2, 201408_at
FIG. 282: PRO84256
FIG. 283: DNA328412, NP_060428.1, 201411_s_at
FIG. 284: PRO84257
FIG. 285: DNA273517, NP_000169.1, 201415_at
FIG. 286: PRO61498
FIG. 287: DNA327550, NP_001959.1, 201435_s_at
FIG. 288: PRO81164
FIG. 289: DNA273396, DNA273396, 201449_at
FIG. 290: DNA325049, NP_005605.1, 201453_x_at
FIG. 291: PRO37938
FIG. 292: DNA274343, NP_000894.1, 201467_s_at
FIG. 293: PRO62259
FIG. 294: DNA328413, NP_004823.1, 201470_at
FIG. 295: PRO84258
FIG. 296: DNA328414, NP_003891.1, 201471_s_at
FIG. 297: PRO81346
FIG. 298: DNA103320, NP_002220.1, 201473_at
FIG. 299: PRO4650
FIG. 300: DNA88608, NP_002893.1, 201485_s_at
FIG. 301: PRO2864
FIG. 302: DNA304459, BC005020, 201489_at
FIG. 303: PRO37073
FIG. 304: DNA304459, NP_005720.1, 201490_s_at
FIG. 305: PRO37073
FIG. 306: DNA253807, NP_065390.1, 201502_s_at
FIG. 307: PRO49210
FIG. 308: DNA328415, BC006997, 201503_at
FIG. 309: PRO60207
FIG. 310: DNA328416, NP_002613.2, 201507_at
FIG. 311: PRO84259
FIG. 312: DNA271931, NP_005745.1, 201514_s_at
FIG. 313: PRO60207
FIG. 314A-B: DNA150463, NP_055635.1, 201519_at
FIG. 315: PRO12269
FIG. 316: DNA328417, ATP6V1F, 201527_at
FIG. 317: PRO84260
FIG. 318: DNA328418, NP_003398.1, 201531_at
FIG. 319: PRO84261
FIG. 320: DNA328419, NP_002779.1, 201532_at
FIG. 321: PRO84262
FIG. 322: DNA328420, BC002682, 201537_s_at
FIG. 323: PRO58245
FIG. 324: DNA88464, NP_005552.2, 201551_s_at
FIG. 325: PRO2804
FIG. 326A-B: DNA290226, NP_039234.1,
201559_s_at
FIG. 327: PRO70317
FIG. 328: DNA227071, NP_000260.1, 201577_at
FIG. 329: PRO37534
FIG. 330A-B: DNA227307, NP_009115.1,
201591_s_at
FIG. 331: PRO37770
FIG. 332: DNA255406, NP_005533.1, 201625_s_at
FIG. 333: PRO50473
FIG. 334A-B: DNA328421, 475621.10, 201646_at
FIG. 335: PRO51048
FIG. 336A-B: DNA220748, NP_000201.1, 201656_at
FIG. 337: PRO34726
FIG. 338: DNA269791, NP_001168.1, 201659_s_at
FIG. 339: PRO58197
FIG. 340A-B: DNA328422, NP_004448.1,
201661_s_at
FIG. 341: PRO84263
FIG. 342: DNA328423, NP_003245.1, 201666_at
FIG. 343: PRO2121
FIG. 344: DNA273090, NP_002347.4, 201670_s_at
FIG. 345: PRO61148
FIG. 346: DNA328424, NP_005142.1, 201672_s_at
FIG. 347: PRO59291
FIG. 348: DNA271223, NP_005070.1, 201689_s_at
FIG. 349: PRO59538
FIG. 350A-B: DNA323965, NP_002848.1,
201706_s_at
FIG. 351: PRO80695
FIG. 352: DNA270883, NP_001061.1, 201714_at
FIG. 353: PRO59218
FIG. 354A-B: DNA328425, NP_065207.2,
201722_s_at
FIG. 355: PRO84264
FIG. 356: DNA328426, NP_000582.1, 201743_at
FIG. 357: PRO384
FIG. 358: DNA150429, NP_002813.1, 201745_at
FIG. 359: PRO12769
FIG. 360: DNA272465, NP_004543.1, 201757_at
FIG. 361: PRO60713
FIG. 362: DNA328427, NP_061109.1, 201760_s_at
FIG. 363: PRO84265
FIG. 364: DNA287167, NP_006627.1, 201761_at
FIG. 365: PRO59136
FIG. 366: DNA323937, NP_005689.2, 201771_at
FIG. 367: PRO80670
FIG. 368: DNA88619, NP_002924.1, 201785_at
FIG. 369: PRO2871
FIG. 370A-B: DNA328428, NP_038479.1,
201798_s_at
FIG. 371: PRO84266
FIG. 372: DNA227563, NP_004946.1, 201801_s_at
FIG. 373: PRO38026
FIG. 374: DNA225896, NP_000109.1, 201808_s_at
FIG. 375: PRO36359
FIG. 376: DNA151017, NP_004835.1, 201810_s_at
FIG. 377: PRO12841
FIG. 378: DNA328429, NP_079106.2, 201818_at
FIG. 379: PRO81201
FIG. 380: DNA328430, NP_005496.2, 201819_at
FIG. 381: PRO84267
FIG. 382: DNA324015, NP_006326.1, 201821_s_at
FIG. 383: PRO80735
FIG. 384: DNA150650, NP_057086.1, 201825_s_at
FIG. 385: PRO12393
FIG. 386: DNA304710, NP_001531.1, 201841_s_at
FIG. 387: PRO71136
FIG. 388: DNA88450, NP_000226.1, 201847_at
FIG. 389: PRO2795
FIG. 390: DNA150725, NP_001738.1, 201850_at
FIG. 391: PRO12792
FIG. 392: DNA272066, NP_002931.1, 201872_s_at
FIG. 393: PRO60337
FIG. 394: DNA328431, NP_001817.1, 201897_s_at
FIG. 395: PRO45093
FIG. 396: DNA103214, NP_006057.1, 201900_s_at
FIG. 397: PRO4544
FIG. 398: DNA227112, NP_006397.1, 201923_at
FIG. 399: PRO37575
FIG. 400: DNA83046, NP_000565.1, 201926_s_at
FIG. 401: PRO2569
FIG. 402: DNA273014, NP_000117.1, 201931_at
FIG. 403: PRO61085
FIG. 404: DNA254147, NP_000512.1, 201944_at
FIG. 405: PRO49262
FIG. 406: DNA274167, NP_006422.1, 201946_s_at
FIG. 407: PRO62097
FIG. 408A-B: DNA327562, HSMEMD, 201951_at
FIG. 409A-B: DNA327563, NP_066945.1, 201963_at
FIG. 410: PRO83592
FIG. 411: DNA227290, NP_055861.1, 201965_s_at
FIG. 412: PRO37753
FIG. 413A-B: DNA328432, NP_005768.1, 201967_at
FIG. 414: PRO61793
FIG. 415A-B: DNA328433, ATP6V1A1,
201971_s_at
FIG. 416: PRO84268
FIG. 417: DNA327073, NP_036418.1, 201994_at
FIG. 418: PRO83365
FIG. 419: DNA226878, NP_000118.1, 201995_at
FIG. 420: PRO37341
FIG. 421A-D: DNA328434, NP_055816.2,
201996_s_at
FIG. 422: PRO84269
FIG. 423: DNA328435, NP_002481.1, 202001_s_at
FIG. 424: PRO60236
FIG. 425: DNA275246, NP_006102.1, 202003_s_at
FIG. 426: PRO62933
FIG. 427: DNA327841, NP_068813.1, 202005_at
FIG. 428: PRO12377
FIG. 429: DNA328436, 1171619.4, 202007_at
FIG. 430: PRO84270
FIG. 431: DNA327564, NP_000111.1, 202017_at
FIG. 432: PRO83593
FIG. 433: DNA328437, AF083441, 202021_x_at
FIG. 434: PRO84271
FIG. 435A-B: DNA270997, NP_005047.1,
202040_s_at
FIG. 436: PRO59326
FIG. 437A-B: DNA327565, NP_056392.1,
202052_s_at
FIG. 438: PRO83594
FIG. 439A-B: DNA327566, NP_000373.1,
202053_s_at
FIG. 440: PRO83595
FIG. 441: DNA226116, NP_002990.1, 202071_at
FIG. 442: PRO36579
FIG. 443A-B: DNA328438, 100983.30, 202073_at
FIG. 444: PRO84272
FIG. 445: DNA328439, NP_068815.1, 202074_s_at
FIG. 446: PRO84273
FIG. 447: DNA290272, NP_004898.1, 202081_at
FIG. 448: PRO70409
FIG. 449: DNA327569, NP_001903.1, 202087_s_at
FIG. 450: PRO2683
FIG. 451: DNA328440, NP_004517.1, 202107_s_at
FIG. 452: PRO84274
FIG. 453: DNA272777, NP_000276.1, 202108_at
FIG. 454: PRO60884
FIG. 455A-B: DNA328441, AL136139, 202149_at
FIG. 456: PRO0
FIG. 457: DNA328442, NP_006078.2, 202154_x_at
FIG. 458: PRO84275
FIG. 459A-C: DNA328443, NP_004371.1, 202160_at
FIG. 460: PRO84276
FIG. 461A-C: DNA271201, NP_005881.1,
202191_s_at
FIG. 462: PRO59518
FIG. 463: DNA328258, SLC16A1, 202236_s_at
FIG. 464: PRO84151
FIG. 465: DNA328444, MGC14458, 202246_s_at
FIG. 466: PRO84277
FIG. 467: DNA294794, NP_002861.1, 202252_at
FIG. 468: PRO70754
FIG. 469A-B: DNA227176, NP_056371.1,
202255_s_at
FIG. 470: PRO37639
FIG. 471: DNA325823, NP_055702.1, 202258_s_at
FIG. 472: PRO82289
FIG. 473: DNA256533, NP_006105.1, 202264_s_at
FIG. 474: PRO51565
FIG. 475: DNA328445, NP_057698.1, 202266_at
FIG. 476: PRO84278
FIG. 477: DNA328446, NP_003896.1, 202268_s_at
FIG. 478: PRO59821
FIG. 479: DNA328447, NP_000393.2, 202275_at
FIG. 480: PRO84279
FIG. 481: DNA304716, NP_510867.1, 202284_s_at
FIG. 482: PRO71142
FIG. 483: DNA270142, NP_005947.2, 202309_at
FIG. 484: PRO58531
FIG. 485: DNA328448, NP_000777.1, 202314_at
FIG. 486: PRO62362
FIG. 487: DNA325115, NP_001435.1, 202345_s_at
FIG. 488: PRO81689
FIG. 489: DNA106239, DNA106239, 202351_at
FIG. 490: DNA270502, NP_002807.1, 202352_s_at
FIG. 491: PRO58880
FIG. 492: DNA327074, FLJ21174, 202371_at
FIG. 493: PRO83366
FIG. 494: DNA149091, DNA149091, 202377_at
FIG. 495A-B: DNA151045, NP_005376.2,
202379_s_at
FIG. 496: PRO12587
FIG. 497A-B: DNA200236, NP_003807.1, 202381_at
FIG. 498: PRO34137
FIG. 499: DNA328449, NP_005462.1, 202382_s_at
FIG. 500: PRO60304
FIG. 501: DNA290234, NP_002914.1, 202388_at
FIG. 502: PRO70333
FIG. 503: DNA269766, NP_005646.1, 202393_s_at
FIG. 504: PRO58175
FIG. 505: DNA227612, NP_056230.1, 202427_s_at
FIG. 506: PRO38075
FIG. 507: DNA324171, NP_065438.1, 202428_x_at
FIG. 508: PRO60753
FIG. 509A-B: DNA327576, NP_000095.1,
202434_s_at
FIG. 510: PRO83600
FIG. 511A-D: DNA328450, NP_077719.1,
202443_x_at
FIG. 512: PRO84280
FIG. 513: DNA225809, NP_000387.1, 202450_s_at
FIG. 514: PRO36272
FIG. 515: DNA227921, NP_003789.1, 202468_s_at
FIG. 516: PRO38384
FIG. 517: DNA150942, HSY18007, 202475_at
FIG. 518: PRO12549
FIG. 519: DNA225566, NP_004744.1, 202481_at
FIG. 520: PRO36029
FIG. 521A-B: DNA103449, NP_008862.1,
202497_x_at
FIG. 522: PRO4776
FIG. 523: DNA328451, NP_000007.1, 202502_at
FIG. 524: PRO62139
FIG. 525A-B: DNA274893, NP_006282.1,
202510_s_at
FIG. 526: PRO62634
FIG. 527: DNA328452, NP_000394.1, 202528_at
FIG. 528: PRO63289
FIG. 529: DNA219229, NP_002189.1, 202531_at
FIG. 530: PRO34544
FIG. 531A-B: DNA274852, NP_004115.1,
202543_s_at
FIG. 532: PRO62605
FIG. 533: DNA328453, NP_003752.2, 202546_at
FIG. 534: PRO84281
FIG. 535A-B: DNA328454, NP_057525.1,
202551_s_at
FIG. 536: PRO4330
FIG. 537: DNA150817, NP_000840.1, 202554_s_at
FIG. 538: PRO12808
FIG. 539: DNA227994, NP_009107.1, 202562_s_at
FIG. 540: PRO38457
FIG. 541: DNA328455, AY007134, 202573_at
FIG. 542: PRO84282
FIG. 543: DNA323923, NP_001869.1, 202575_at
FIG. 544: PRO80657
FIG. 545: DNA328456, NP_000467.1, 202587_s_at
FIG. 546: PRO84283
FIG. 547: DNA328457, NP_036422.1, 202606_s_at
FIG. 548: PRO70421
FIG. 549: DNA103245, NP_002341.1, 202626_s_at
FIG. 550: PRO4575
FIG. 551: DNA83141, NP_000593.1, 202627_s_at
FIG. 552: PRO2604
FIG. 553: DNA254129, NP_006001.1, 202655_at
FIG. 554: PRO49244
FIG. 555: DNA270379, NP_002792.1, 202659_at
FIG. 556: PRO58763
FIG. 557: DNA326896, NP_003672.1, 202671_s_at
FIG. 558: PRO69486
FIG. 559: DNA289526, NP_004015.2, 202672_s_at
FIG. 560: PRO70282
FIG. 561: DNA273542, NP_002991.1, 202675_at
FIG. 562: PRO61522
FIG. 563: DNA328458, NP_037458.2, 202679_at
FIG. 564: PRO84284
FIG. 565: DNA84130, NP_003801.1, 202687_s_at
FIG. 566: PRO1096
FIG. 567: DNA271085, NP_004751.1, 202693_s_at
FIG. 568: PRO59409
FIG. 569A-B: DNA150467, NP_055513.1,
202699_s_at
FIG. 570: PRO12272
FIG. 571A-B: DNA328459, NP_004332.2, 202715_at
FIG. 572: PRO84285
FIG. 573: DNA273290, NP_002047.1, 202722_s_at
FIG. 574: PRO61300
FIG. 575: DNA328460, NP_004190.1, 202733_at
FIG. 576: PRO84286
FIG. 577: DNA150713, NP_006570.1, 202735_at
FIG. 578: PRO12082
FIG. 579A-B: DNA328461, 350230.2, 202741_at
FIG. 580: PRO84287
FIG. 581: DNA271973, NP_002722.1, 202742_s_at
FIG. 582: PRO60248
FIG. 583A-B: DNA150943, NP_036376.1,
202752_x_at
FIG. 584: PRO12550
FIG. 585A-C: DNA328462, HSA303079,
202759_s_at
FIG. 586: PRO84288
FIG. 587A-C: DNA328463, NP_009134.1,
202760_s_at
FIG. 588: PRO84289
FIG. 589: DNA226080, NP_001601.1, 202767_at
FIG. 590: PRO36543
FIG. 591A-B: DNA150977, NP_006723.1, 202768_at
FIG. 592: PRO12828
FIG. 593A-B: DNA328464, 977954.20, 202769_at
FIG. 594: PRO84290
FIG. 595: DNA226578, NP_004345.1, 202770_s_at
FIG. 596: PRO37041
FIG. 597A-B: DNA103521, NP_004163.1, 202800_at
FIG. 598: PRO4848
FIG. 599A-B: DNA327583, ABCC1, 202805_s_at
FIG. 600: PRO83604
FIG. 601: DNA328465, NP_005639.1, 202823_at
FIG. 602: PRO84291
FIG. 603: DNA225865, NP_004986.1, 202827_s_at
FIG. 604: PRO36328
FIG. 605: DNA225926, NP_000138.1, 202838_at
FIG. 606: PRO36389
FIG. 607: DNA328466, NP_004554.1, 202847_at
FIG. 608: PRO84292
FIG. 609: DNA103394, NP_004198.1, 202855_s_at
FIG. 610: PRO4722
FIG. 611: DNA275144, NP_000128.1, 202862_at
FIG. 612: PRO62852
FIG. 613: DNA328467, SP100, 202864_s_at
FIG. 614: PRO84293
FIG. 615: DNA287289, NP_058132.1, 202869_at
FIG. 616: PRO69559
FIG. 617: DNA328468, BC010960, 202872_at
FIG. 618: PRO84294
FIG. 619: DNA328469, NP_001686.1, 202874_s_at
FIG. 620: PRO84295
FIG. 621A-B: DNA255318, NP_036204.1,
202877_s_at
FIG. 622: PRO50388
FIG. 623A-B: DNA328470, NP_055620.1, 202909_at
FIG. 624: PRO84296
FIG. 625: DNA327584, NP_002955.2, 202917_s_at
FIG. 626: PRO80649
FIG. 627: DNA272425, NP_001489.1, 202923_s_at
FIG. 628: PRO60677
FIG. 629: DNA328471, ZMPSTE24, 202939_at
FIG. 630: PRO84297
FIG. 631: DNA269481, NP_001976.1, 202942_at
FIG. 632: PRO57901
FIG. 633: DNA328472, NP_000482.2, 202953_at
FIG. 634: PRO84298
FIG. 635A-B: DNA328473, NP_006473.1,
202968_s_at
FIG. 636: PRO84299
FIG. 637A-C: DNA328474, 1501914.1, 202969_at
FIG. 638: PRO84300
FIG. 639: DNA325915, ZAP128, 202982_s_at
FIG. 640: PRO82369
FIG. 641: DNA271272, NP_000366.1, 203031_s_at
FIG. 642: PRO59583
FIG. 643: DNA324049, FH, 203032_s_at
FIG. 644: PRO62607
FIG. 645A-B: DNA271865, NP_055566.1,
203037_s_at
FIG. 646: PRO60145
FIG. 647: DNA328475, LAMP2, 203042_at
FIG. 648: PRO84301
FIG. 649A-B: DNA328476, AF074331, 203058_s_at
FIG. 650: PRO84302
FIG. 651: DNA256830, NP_004815.1, 203100_s_at
FIG. 652: PRO51761
FIG. 653: DNA272867, NP_003960.1, 203109_at
FIG. 654: PRO60960
FIG. 655A-B: DNA227582, NP_000608.1,
203124_s_at
FIG. 656: PRO38045
FIG. 657: DNA328477, NP_003767.1, 203152_at
FIG. 658: PRO84303
FIG. 659A-B: DNA328478, NP_055720.2,
203158_s_at
FIG. 660: PRO84304
FIG. 661: DNA226136, NP_003246.1, 203167_at
FIG. 662: PRO36599
FIG. 663: DNA328479, NP_001473.1, 203178_at
FIG. 664: PRO84305
FIG. 665A-C: DNA328480, NP_001990.1, 203184_at
FIG. 666: PRO84306
FIG. 667A-B: DNA271010, NP_055552.1, 203185_at
FIG. 668: PRO59339
FIG. 669: DNA270448, NP_002487.1, 203189_s_at
FIG. 670: PRO58827
FIG. 671A-B: DNA328481, MTMR2, 203211_s_at
FIG. 672: PRO84307
FIG. 673A-C: DNA328482, NP_000426.1,
203238_s_at
FIG. 674: PRO84308
FIG. 675: DNA328483, NP_061163.1, 203255_at
FIG. 676: PRO84309
FIG. 677: DNA227127, NP_003571.1, 203269_at
FIG. 678: PRO37590
FIG. 679: DNA328484, UNC119, 203271_s_at
FIG. 680: PRO84310
FIG. 681: DNA302020, NP_005564.1, 203276_at
FIG. 682: PRO70993
FIG. 683A-B: DNA328485, BHC80, 203278_s_at
FIG. 684: PRO84311
FIG. 685: DNA328486, NP_000149.1, 203282_at
FIG. 686: PRO60119
FIG. 687: DNA328487, AF251295, 203299_s_at
FIG. 688: PRO84312
FIG. 689: DNA328488, NP_003907.2, 203300_x_at
FIG. 690: PRO84313
FIG. 691: DNA328489, NP_006511.1, 203303_at
FIG. 692: PRO84314
FIG. 693A-B: DNA328490, NP_000120.1, 203305_at
FIG. 694: PRO84315
FIG. 695: DNA327593, NP_006205.1, 203335_at
FIG. 696: PRO59733
FIG. 697: DNA328491, ICAP-1A, 203336_s_at
FIG. 698: PRO61323
FIG. 699A-B: DNA328492, NP_056125.1,
203354_s_at
FIG. 700: PRO84316
FIG. 701: DNA328493, NP_008957.1, 203367_at
FIG. 702: PRO84317
FIG. 703: DNA328494, RPS6KA1, 203379_at
FIG. 704: PRO84318
FIG. 705: DNA274960, NP_008856.1, 203380_x_at
FIG. 706: PRO62694
FIG. 707: DNA88084, NP_000032.1, 203381_s_at
FIG. 708: PRO2644
FIG. 709A-B: DNA254616, NP_004473.1,
203397_s_at
FIG. 710: PRO49718
FIG. 711: DNA326892, NP_003711.1, 203405_at
FIG. 712: PRO83213
FIG. 713: DNA323927, NP_005563.1, 203411_s_at
FIG. 714: PRO80660
FIG. 715: DNA151037, NP_036461.1, 203414_at
FIG. 716: PRO12586
FIG. 717: DNA273410, NP_004036.1, 203454_s_at
FIG. 718: PRO61409
FIG. 719: DNA328495, NP_055578.1, 203465_at
FIG. 720: PRO58967
FIG. 721: DNA328496, NP_002428.1, 203466_at
FIG. 722: PRO80786
FIG. 723A-B: DNA255622, NP_009187.1,
203472_s_at
FIG. 724: PRO50686
FIG. 725A-C: DNA328497, NP_005493.1,
203504_s_at
FIG. 726: PRO84319
FIG. 727A-C: DNA328498, AF285167, 203505_at
FIG. 728: PRO84320
FIG. 729A-B: DNA188400, NP_001057.1, 203508_at
FIG. 730: PRO21928
FIG. 731A-B: DNA328499, NP_003096.1, 203509_at
FIG. 732: PRO84321
FIG. 733: DNA272911, NP_006545.1, 203517_at
FIG. 734: PRO60997
FIG. 735A-D: DNA328500, NP_000072.1,
203518_at
FIG. 736: PRO84322
FIG. 737A-B: DNA103296, NP_006369.1, 203528_at
FIG. 738: PRO4626
FIG. 739: DNA323910, NP_002956.1, 203535_at
FIG. 740: PRO80648
FIG. 741A-B: DNA272399, NP_001197.1,
203543_s_at
FIG. 742: PRO60653
FIG. 743: DNA328501, NP_076984.1, 203545_at
FIG. 744: PRO84323
FIG. 745: DNA88453, NP_000228.1, 203548_s_at
FIG. 746: PRO2797
FIG. 747: DNA328502, NP_006566.2, 203553_s_at
FIG. 748: PRO84324
FIG. 749: DNA328503, NP_000272.1, 203557_s_at
FIG. 750: PRO10850
FIG. 751: DNA327594, NP_003869.1, 203560_at
FIG. 752: PRO83611
FIG. 753: DNA225916, NP_067674.1, 203561_at
FIG. 754: PRO36379
FIG. 755: DNA273676, NP_055488.1, 203584_at
FIG. 756: PRO61644
FIG. 757: DNA83085, NP_000751.1, 203591_s_at
FIG. 758: PRO2583
FIG. 759: DNA271003, NP_003720.1, 203594_at
FIG. 760: PRO59332
FIG. 761A-B: DNA328504, 1400155.1, 203608_at
FIG. 762: PRO84325
FIG. 763: DNA328505, NP_002484.1, 203613_s_at
FIG. 764: PRO62117
FIG. 765: DNA328506, NP_001046.1, 203615_x_at
FIG. 766: PRO84326
FIG. 767: DNA225774, NP_005079.1, 203624_at
FIG. 768: PRO36237
FIG. 769: DNA254642, NP_004100.1, 203646_at
FIG. 770: PRO49743
FIG. 771: DNA328507, NP_006395.1, 203650_at
FIG. 772: PRO4761
FIG. 773A-B: DNA272998, NP_055548.1, 203651_at
FIG. 774: PRO61070
FIG. 775: DNA328508, NP_003368.1, 203683_s_at
FIG. 776: PRO35975
FIG. 777: DNA255298, NP_004394.1, 203695_s_at
FIG. 778: PRO50371
FIG. 779: DNA227020, NP_001416.1, 203729_at
FIG. 780: PRO37483
FIG. 781: DNA328509, NP_006739.1, 203760_s_at
FIG. 782: PRO57996
FIG. 783: DNA328510, NP_055066.1, 203775_at
FIG. 784: PRO84327
FIG. 785A-B: DNA194602, NP_006370.1,
203789_s_at
FIG. 786: PRO23944
FIG. 787: DNA328511, NP_031397.1, 203825_at
FIG. 788: PRO57838
FIG. 789A-B: DNA328512, NP_005772.2,
203839_s_at
FIG. 790: PRO84328
FIG. 791A-B: DNA272451, HSU86453, 203879_at
FIG. 792: PRO60700
FIG. 793: DNA82429, NP_003011.1, 203889_at
FIG. 794: PRO2558
FIG. 795: DNA328513, NP_057367.1, 203893_at
FIG. 796: PRO37815
FIG. 797: DNA150974, NP_005684.1, 203920_at
FIG. 798: PRO12224
FIG. 799: DNA271676, NP_002052.1, 203925_at
FIG. 800: PRO59961
FIG. 801: DNA88239, NP_004985.1, 203936_s_at
FIG. 802: PRO2711
FIG. 803: DNA227232, NP_001850.1, 203971_at
FIG. 804: PRO37695
FIG. 805: DNA328514, NP_005186.1, 203973_s_at
FIG. 806: PRO84329
FIG. 807: DNA328515, NP_000775.1, 203979_at
FIG. 808: PRO84330
FIG. 809: DNA327608, NP_001433.1, 203980_at
FIG. 810: PRO83617
FIG. 811: DNA328516, NP_005833.1, 204011_at
FIG. 812: PRO12323
FIG. 813: DNA328517, NP_003558.1, 204032_at
FIG. 814: PRO84331
FIG. 815: DNA226342, NP_000305.1, 204054_at
FIG. 816: PRO36805
FIG. 817: DNA327609, 1448428.2, 204058_at
FIG. 818: PRO83618
FIG. 819: DNA328518, ME1, 204059_s_at
FIG. 820: PRO84332
FIG. 821: DNA226737, NP_004576.1, 204070_at
FIG. 822: PRO37200
FIG. 823A-C: DNA328519, NP_075463.1,
204072_s_at
FIG. 824: PRO84333
FIG. 825: DNA328520, NP_079353.1, 204080_at
FIG. 826: PRO84334
FIG. 827A-B: DNA150739, NP_006484.1,
204084_s_at
FIG. 828: PRO12442
FIG. 829: DNA227130, NP_002551.1, 204088_at
FIG. 830: PRO37593
FIG. 831: DNA328521, NP_003069.1, 204099_at
FIG. 832: PRO62553
FIG. 833: DNA328522, NP_001769.2, 204118_at
FIG. 834: PRO2696
FIG. 835: DNA328523, NP_006712.1, 204119_s_at
FIG. 836: PRO84335
FIG. 837: DNA328524, NP_057097.1, 204125_at
FIG. 838: PRO84336
FIG. 839: DNA328525, BC021224, 204131_s_at
FIG. 840: PRO84337
FIG. 841: DNA103532, NP_003263.1, 204137_at
FIG. 842: PRO4859
FIG. 843: DNA324816, NP_001060.1, 204141_at
FIG. 844: PRO81429
FIG. 845: DNA270524, NP_059982.1, 204142_at
FIG. 846: PRO58901
FIG. 847: DNA328526, NP_000841.1, 204149_s_at
FIG. 848: PRO37856
FIG. 849A-B: DNA150497, DNA150497,
204155_s_at
FIG. 850: PRO12296
FIG. 851A-B: DNA328527, NP_055751.1,
204160_s_at
FIG. 852: PRO4351
FIG. 853: DNA328528, MLC1SA, 204173_at
FIG. 854: PRO60636
FIG. 855: DNA328529, NP_001620.2, 204174_at
FIG. 856: PRO49814
FIG. 857: DNA226380, NP_001765.1, 204192_at
FIG. 858: PRO4695
FIG. 859: DNA273070, NP_005189.2, 204193_at
FIG. 860: PRO70107
FIG. 861: DNA227514, NP_000152.1, 204224_s_at
FIG. 862: PRO37977
FIG. 863: DNA270434, NP_006434.1, 204238_s_at
FIG. 864: PRO58814
FIG. 865: DNA307936, NP_004926.1, 204247_s_at
FIG. 866: PRO71356
FIG. 867A-B: DNA188734, NP_001261.1, 204258_at
FIG. 868: PRO22296
FIG. 869: DNA226577, NP_071390.1, 204265_s_at
FIG. 870: PRO37040
FIG. 871: DNA273802, NP_066950.1, 204285_s_at
FIG. 872: PRO61763
FIG. 873: DNA328530, NP_009198.2, 204328_at
FIG. 874: PRO24118
FIG. 875: DNA328531, NP_037542.1, 204348_s_at
FIG. 876: PRO84338
FIG. 877: DNA328532, LIMK1, 204357_s_at
FIG. 878: PRO84339
FIG. 879: DNA225750, NP_000254.1, 204360_s_at
FIG. 880: PRO36213
FIG. 881: DNA328533, NP_003647.1, 204392_at
FIG. 882: PRO84340
FIG. 883: DNA272469, NP_005299.1, 204396_s_at
FIG. 884: PRO60717
FIG. 885: DNA226462, NP_002241.1, 204401_at
FIG. 886: PRO36925
FIG. 887: DNA225756, NP_001636.1, 204416_x_at
FIG. 888: PRO36219
FIG. 889: DNA226286, NP_001657.1, 204425_at
FIG. 890: PRO36749
FIG. 891A-B: DNA88476, NP_002429.1, 204438_at
FIG. 892: PRO2811
FIG. 893: DNA150972, NP_005252.1, 204472_at
FIG. 894: PRO12162
FIG. 895: DNA194652, NP_001187.1, 204493_at
FIG. 896: PRO23974
FIG. 897: DNA328534, NP_056307.1, 204494_s_at
FIG. 898: PRO84341
FIG. 899: DNA328254, BC002678, 204517_at
FIG. 900: PRO11581
FIG. 901: DNA328254, NP_000934.1, 204518_s_at
FIG. 902: PRO11581
FIG. 903A-B: DNA328535, NP_009147.1, 204544_at
FIG. 904: PRO60044
FIG. 905: DNA225993, NP_000646.1, 204563_at
FIG. 906: PRO36456
FIG. 907: DNA287284, NP_060943.1, 204565_at
FIG. 908: PRO59915
FIG. 909: DNA151910, NP_004906.2, 204567_s_at
FIG. 910: PRO12754
FIG. 911: DNA270564, NP_004499.1, 204615_x_at
FIG. 912: PRO58939
FIG. 913: DNA328536, 1099945.20, 204619_s_at
FIG. 914: PRO84342
FIG. 915A-D: DNA328537, NP_004376.2,
204620_s_at
FIG. 916: PRO84343
FIG. 917: DNA151048, NP_006177.1, 204621_s_at
FIG. 918: PRO12850
FIG. 919A-B: DNA328538, 351122.2, 204627_s_at
FIG. 920: PRO84344
FIG. 921A-B: DNA88429, NP_000203.1,
204628_s_at
FIG. 922: PRO2344
FIG. 923: DNA226079, NP_001602.1, 204638_at
FIG. 924: PRO36542
FIG. 925: DNA272078, NP_003019.1, 204657_s_at
FIG. 926: PRO60348
FIG. 927: DNA227425, NP_001038.1, 204675_at
FIG. 928: PRO37888
FIG. 929A-B: DNA328539, NP_000121.1,
204713_s_at
FIG. 930: PRO84345
FIG. 931: DNA328540, NP_006144.1, 204725_s_at
FIG. 932: PRO12168
FIG. 933A-B: DNA325192, NP_038203.1,
204744_s_at
FIG. 934: PRO81753
FIG. 935: DNA328541, NP_004503.1, 204773_at
FIG. 936: PRO4843
FIG. 937: DNA328542, NP_055025.1, 204774_at
FIG. 938: PRO2577
FIG. 939: DNA327050, NP_009199.1, 204787_at
FIG. 940: PRO34043
FIG. 941: DNA328543, NP_005883.1, 204789_at
FIG. 942: PRO84346
FIG. 943: DNA272121, NP_005895.1, 204790_at
FIG. 944: PRO60391
FIG. 945: DNA324799, NP_061823.1, 204806_x_at
FIG. 946: PRO81414
FIG. 947: DNA154704, DNA154704, 204807_at
FIG. 948: DNA328544, NP_006673.1, 204834_at
FIG. 949: PRO84347
FIG. 950: DNA225661, NP_001944.1, 204858_s_at
FIG. 951: PRO36124
FIG. 952: DNA328545, NP_064525.1, 204859_s_at
FIG. 953: PRO84348
FIG. 954A-B: DNA227629, NP_004527.1,
204860_s_at
FIG. 955: PRO38092
FIG. 956: DNA328546, NP_005249.1, 204867_at
FIG. 957: PRO84349
FIG. 958: DNA255993, NP_008936.1, 204872_at
FIG. 959: PRO51044
FIG. 960: DNA273666, NP_003349.1, 204881_s_at
FIG. 961: PRO61634
FIG. 962A-B: DNA76503, NP_001549.1, 204912_at
FIG. 963: PRO2536
FIG. 964: DNA328547, TLR2, 204924_at
FIG. 965: PRO208
FIG. 966: DNA228014, NP_002153.1, 204949_at
FIG. 967: PRO38477
FIG. 968: DNA328548, NP_006298.1, 204955_at
FIG. 969: PRO2618
FIG. 970: DNA103283, NP_002423.1, 204959_at
FIG. 971: PRO4613
FIG. 972: DNA227091, NP_000256.1, 204961_s_at
FIG. 973: PRO37554
FIG. 974A-B: DNA328549, NP_002897.1,
204969_s_at
FIG. 975: PRO84350
FIG. 976: DNA328301, NP_005204.1, 204971_at
FIG. 977: PRO70371
FIG. 978A-B: DNA328550, NP_001439.2,
204983_s_at
FIG. 979: PRO937
FIG. 980: DNA269665, NP_002454.1, 204994_at
FIG. 981: PRO58076
FIG. 982A-B: DNA273686, NP_055520.1, 205003_at
FIG. 983: PRO61653
FIG. 984: DNA272427, NP_004799.1, 205005_s_at
FIG. 985: PRO60679
FIG. 986: DNA194830, NP_055437.1, 205011_at
FIG. 987: PRO24094
FIG. 988: DNA328551, NP_003823.1, 205048_s_at
FIG. 989: PRO84351
FIG. 990A-B: DNA328552, NP_055886.1,
205068_s_at
FIG. 991: PRO84352
FIG. 992: DNA328553, NP_061944.1, 205070_at
FIG. 993: PRO84353
FIG. 994: DNA194627, NP_003051.1, 205074_at
FIG. 995: PRO23962
FIG. 996: DNA272181, NP_006688.1, 205076_s_at
FIG. 997: PRO60446
FIG. 998: DNA254216, NP_002020.1, 205119_s_at
FIG. 999: PRO49328
FIG. 1000: DNA299899, NP_002148.1, 205133_s_at
FIG. 1001: PRO62760
FIG. 1002: DNA328554, NP_038202.1, 205147_x_at
FIG. 1003: PRO84354
FIG. 1004: DNA328555, NP_001241.1, 205153_s_at
FIG. 1005: PRO34457
FIG. 1006: DNA80896, NP_001100.1, 205180_s_at
FIG. 1007: PRO1686
FIG. 1008: DNA328556, NP_004568.1, 205194_s_at
FIG. 1009: PRO84355
FIG. 1010: DNA273535, NP_004217.1, 205214_at
FIG. 1011: PRO61515
FIG. 1012: DNA93504, NP_006009.1, 205220_at
FIG. 1013: PRO4923
FIG. 1014: DNA325255, NP_001994.2, 205237_at
FIG. 1015: PRO1910
FIG. 1016: DNA327634, NP_005129.1, 205241_at
FIG. 1017: PRO83636
FIG. 1018: DNA227081, NP_000390.2, 205249_at
FIG. 1019: PRO37544
FIG. 1020: DNA328557, NP_001098.1, 205260_s_at
FIG. 1021: PRO84356
FIG. 1022: DNA328558, BC016618, 205269_at
FIG. 1023: PRO84357
FIG. 1024: DNA328559, NP_005556.1, 205270_s_at
FIG. 1025: PRO84358
FIG. 1026A-B: DNA227505, NP_003670.1,
205306_x_at
FIG. 1027: PRO37968
FIG. 1028: DNA325783, NP_002558.1, 205353_s_at
FIG. 1029: PRO59001
FIG. 1030: DNA88215, NP_001919.1, 205382_s_at
FIG. 1031: PRO2703
FIG. 1032: DNA328560, NP_003650.1, 205401_at
FIG. 1033: PRO84359
FIG. 1034: DNA328561, NP_004624.1, 205403_at
FIG. 1035: PRO2019
FIG. 1036: DNA327638, NP_005516.1, 205404_at
FIG. 1037: PRO83639
FIG. 1038: DNA328562, NP_000010.1, 205412_at
FIG. 1039: PRO84360
FIG. 1040A-B: DNA328563, NP_005329.2,
205425_at
FIG. 1041: PRO81554
FIG. 1042: DNA328564, HPCAL1, 205462_s_at
FIG. 1043: PRO84361
FIG. 1044: DNA196825, NP_005105.1, 205466_s_at
FIG. 1045: PRO25266
FIG. 1046: DNA328565, NP_057070.1, 205474_at
FIG. 1047: PRO84362
FIG. 1048: DNA226153, NP_002649.1, 205479_s_at
FIG. 1049: PRO36616
FIG. 1050: DNA287224, NP_005092.1, 205483_s_at
FIG. 1051: PRO69503
FIG. 1052: DNA328566, NP_060446.1, 205510_s_at
FIG. 1053: PRO84363
FIG. 1054: DNA328567, NP_006797.2, 205548_s_at
FIG. 1055: PRO84364
FIG. 1056: DNA227535, NP_066190.1, 205568_at
FIG. 1057: PRO37998
FIG. 1058A-B: DNA327643, NP_055712.1,
205594_at
FIG. 1059: PRO83644
FIG. 1060A-C: DNA328568, NP_006720.1,
205603_s_at
FIG. 1061: PRO59731
FIG. 1062: DNA324324, NP_000679.1, 205633_s_at
FIG. 1063: PRO81000
FIG. 1064: DNA328569, NP_077274.1, 205634_x_at
FIG. 1065: PRO84365
FIG. 1066: DNA88076, NP_001628.1, 205639_at
FIG. 1067: PRO2640
FIG. 1068: DNA287317, NP_003724.1, 205660_at
FIG. 1069: PRO69582
FIG. 1070: DNA328570, NP_004040.1, 205681_at
FIG. 1071: PRO37843
FIG. 1072: DNA327644, NP_060395.2, 205684_s_at
FIG. 1073: PRO83645
FIG. 1074: DNA150621, NP_036595.1, 205704_s_at
FIG. 1075: PRO12374
FIG. 1076: DNA328571, NP_001254.1, 205709_s_at
FIG. 1077: PRO84366
FIG. 1078: DNA88106, NP_004325.1, 205715_at
FIG. 1079: PRO2655
FIG. 1080: DNA270401, NP_003140.1, 205743_at
FIG. 1081: PRO58784
FIG. 1082: DNA275620, NP_000628.1, 205770_at
FIG. 1083: PRO63244
FIG. 1084: DNA88187, NP_001757.1, 205789_at
FIG. 1085: PRO2689
FIG. 1086: DNA76517, NP_002176.1, 205798_at
FIG. 1087: PRO2541
FIG. 1088A-B: DNA271915, NP_056191.1,
205801_s_at
FIG. 1089: PRO60192
FIG. 1090: DNA194766, NP_079504.1, 205804_s_at
FIG. 1091: PRO24046
FIG. 1092: DNA328572, NP_004309.2, 205808_at
FIG. 1093: PRO84367
FIG. 1094: DNA328573, NP_006761.1, 205819_at
FIG. 1095: PRO1559
FIG. 1096A-B: DNA328574, NP_004963.1,
205842_s_at
FIG. 1097: PRO84368
FIG. 1098: DNA327651, NP_005612.1, 205863_at
FIG. 1099: PRO83649
FIG. 1100: DNA328575, NP_071754.2, 205872_x_at
FIG. 1101: PRO84369
FIG. 1102A-B: DNA220746, NP_000876.1,
205884_at
FIG. 1103: PRO34724
FIG. 1104A-B: DNA273962, NP_055605.1,
205888_s_at
FIG. 1105: PRO61910
FIG. 1106: DNA93423, NP_000667.1, 205891_at
FIG. 1107: PRO4944
FIG. 1108: DNA328576, HSU20350, 205898_at
FIG. 1109: PRO4940
FIG. 1110: DNA328577, NP_003905.1, 205899_at
FIG. 1111: PRO59588
FIG. 1112A-B: DNA196549, NP_003034.1,
205920_at
FIG. 1113: PRO25031
FIG. 1114: DNA328578, NP_004656.2, 205922_at
FIG. 1115: PRO7426
FIG. 1116A-B: DNA270867, NP_006217.1,
205934_at
FIG. 1117: PRO59203
FIG. 1118: DNA76516, NP_000556.1, 205945_at
FIG. 1119: PRO2022
FIG. 1120: DNA196439, NP_003865.1, 205988_at
FIG. 1121: PRO24934
FIG. 1122: DNA36722, NP_000576.1, 205992_s_at
FIG. 1123: PRO77
FIG. 1124: DNA328579, BC020082, 206020_at
FIG. 1125: PRO84370
FIG. 1126: DNA328580, HSU27699, 206058_at
FIG. 1127: PRO4627
FIG. 1128: DNA328581, NP_002122.1, 206074_s_at
FIG. 1129: PRO34536
FIG. 1130: DNA328582, NP_001865.1, 206100_at
FIG. 1131: PRO84371
FIG. 1132: DNA226105, NP_002925.1, 206111_at
FIG. 1133: PRO36568
FIG. 1134: DNA225764, NP_000037.1, 206129_s_at
FIG. 1135: PRO36227
FIG. 1136: DNA328583, ASGR2, 206130_s_at
FIG. 1137: PRO84372
FIG. 1138: DNA327656, NP_055294.1, 206134_at
FIG. 1139: PRO36117
FIG. 1140A-B: DNA271837, NP_055497.1,
206135_at
FIG. 1141: PRO60117
FIG. 1142: DNA328584, NP_001148.1, 206200_s_at
FIG. 1143: PRO4833
FIG. 1144: DNA226058, NP_005075.1, 206214_at
FIG. 1145: PRO36521
FIG. 1146: DNA218691, NP_003832.1, 206222_at
FIG. 1147: PRO34469
FIG. 1148A-C: DNA328585, AF286028,
206239_s_at
FIG. 1149: DNA328586, NP_002369.2, 206267_s_at
FIG. 1150: PRO84373
FIG. 1151: DNA328587, NP_002612.1, 206380_s_at
FIG. 1152: PRO2854
FIG. 1153: DNA255814, NP_005840.1, 206420_at
FIG. 1154: PRO50869
FIG. 1155: DNA328588, NP_060823.1, 206500_s_at
FIG. 1156: PRO84374
FIG. 1157: DNA270444, NP_004824.1, 206513_at
FIG. 1158: PRO58823
FIG. 1159: DNA196614, NP_001158.1, 206536_s_at
FIG. 1160: PRO25091
FIG. 1161: DNA270019, NP_036351.1, 206538_at
FIG. 1162: PRO58414
FIG. 1163: DNA327663, NP_006771.1, 206565_x_at
FIG. 1164: PRO83654
FIG. 1165: DNA327665, NP_002099.1, 206643_at
FIG. 1166: PRO83655
FIG. 1167: DNA328589, BCL2L1, 206665_s_at
FIG. 1168: PRO83141
FIG. 1169: DNA328590, C6orf32, 206707_x_at
FIG. 1170: PRO84375
FIG. 1171A-B: DNA88191, NP_001234.1, 206729_at
FIG. 1172: PRO2691
FIG. 1173: DNA327669, NP_000914.1, 206792_x_at
FIG. 1174: PRO83657
FIG. 1175: DNA270107, NP_006856.1, 206881_s_at
FIG. 1176: PRO58498
FIG. 1177: DNA256561, NP_062550.1, 206914_at
FIG. 1178: PRO51592
FIG. 1179: DNA328591, NP_006635.1, 206976_s_at
FIG. 1180: PRO84376
FIG. 1181A-B: DNA227659, NP_000570.1,
206991_s_at
FIG. 1182: PRO38122
FIG. 1183: DNA188289, NP_001548.1, 207008_at
FIG. 1184: PRO21820
FIG. 1185: DNA328592, AB015228, 207016_s_at
FIG. 1186: PRO84377
FIG. 1187: DNA227531, NP_004722.1, 207057_at
FIG. 1188: PRO37994
FIG. 1189: DNA327673, NP_002188.1, 207071_s_at
FIG. 1190: PRO83660
FIG. 1191A-B: DNA328593, CLAS1, 207075_at
FIG. 1192: PRO84378
FIG. 1193A-B: DNA328594, CSF1, 207082_at
FIG. 1194: PRO84379
FIG. 1195: DNA88291, NP_001965.1, 207111_at
FIG. 1196: PRO2729
FIG. 1197A-B: DNA327674, NP_002739.1,
207121_s_at
FIG. 1198: PRO83661
FIG. 1199: DNA328595, NP_001045.1, 207122_x_at
FIG. 1200: PRO84380
FIG. 1201: DNA226996, NP_000239.1, 207233_s_at
FIG. 1202: PRO37459
FIG. 1203A-B: DNA226536, NP_003225.1,
207332_s_at
FIG. 1204: PRO36999
FIG. 1205: DNA227668, NP_000158.1, 207387_s_at
FIG. 1206: PRO38131
FIG. 1207: DNA328596, DEGS, 207431_s_at
FIG. 1208: PRO37741
FIG. 1209: DNA274829, NP_003653.1, 207469_s_at
FIG. 1210: PRO62588
FIG. 1211: DNA328597, NP_001680.1, 207507_s_at
FIG. 1212: PRO84381
FIG. 1213: DNA328598, NP_055146.1, 207528_s_at
FIG. 1214: PRO23276
FIG. 1215: DNA328599, NFKB2, 207535_s_at
FIG. 1216: PRO84382
FIG. 1217: DNA328600, NP_004839.1, 207571_x_at
FIG. 1218: PRO84383
FIG. 1219: DNA328601, NP_056490.1, 207574_s_at
FIG. 1220: PRO84384
FIG. 1221: DNA328602, NP_002261.1, 207657_x_at
FIG. 1222: PRO84385
FIG. 1223: DNA226278, NP_005865.1, 207697_x_at
FIG. 1224: PRO36741
FIG. 1225: DNA227395, NP_005331.1, 207721_x_at
FIG. 1226: PRO37858
FIG. 1227: DNA325654, NP_054752.1, 207761_s_at
FIG. 1228: PRO4348
FIG. 1229: DNA226930, NP_004152.1, 207791_s_at
FIG. 1230: PRO37393
FIG. 1231: DNA328603, NP_000304.1, 207808_s_at
FIG. 1232: PRO84386
FIG. 1233: DNA328604, NP_001174.2, 207809_s_at
FIG. 1234: PRO84387
FIG. 1235: DNA327682, NP_001905.1, 207843_x_at
FIG. 1236: PRO83666
FIG. 1237: DNA36708, NP_002081.1, 207850_at
FIG. 1238: PRO34256
FIG. 1239: DNA199788, NP_002981.1, 207861_at
FIG. 1240: PRO34107
FIG. 1241: DNA328605, ST7, 207871_s_at
FIG. 1242: PRO84388
FIG. 1243: DNA256523, NP_006854.1, 207872_s_at
FIG. 1244: PRO51557
FIG. 1245: DNA218651, NP_003798.1, 207907_at
FIG. 1246: PRO34447
FIG. 1247: DNA275286, NP_009205.1, 208002_s_at
FIG. 1248: PRO62967
FIG. 1249A-B: DNA328606, CBFA2T3, 208056_s_at
FIG. 1250: PRO84389
FIG. 1251A-B: DNA328607, NP_003639.1,
208072_s_at
FIG. 1252: PRO84390
FIG. 1253: DNA327685, NP_067586.1, 208074_s_at
FIG. 1254: PRO83669
FIG. 1255: DNA328608, NP_006264.2, 208075_s_at
FIG. 1256: PRO9932
FIG. 1257: DNA255376, NP_110423.1, 208091_s_at
FIG. 1258: PRO50444
FIG. 1259: DNA327686, NP_005898.1, 208116_s_at
FIG. 1260: PRO83670
FIG. 1261A-B: DNA328609, NP_109592.1,
208121_s_at
FIG. 1262: PRO84391
FIG. 1263: DNA328610, NP_112601.1, 208146_s_at
FIG. 1264: PRO84392
FIG. 1265A-B: DNA226706, NP_003777.2,
208161_s_at
FIG. 1266: PRO37169
FIG. 1267: DNA328611, RASGRP2, 208206_s_at
FIG. 1268: PRO84393
FIG. 1269: DNA328612, NP_000166.2, 208308_s_at
FIG. 1270: PRO84394
FIG. 1271: DNA270558, NP_006734.1, 208319_s_at
FIG. 1272: PRO58933
FIG. 1273: DNA227614, NP_004859.1, 208336_s_at
FIG. 1274: PRO38077
FIG. 1275: DNA327690, NP_004022.1, 208436_s_at
FIG. 1276: PRO83673
FIG. 1277: DNA328613, NP_056953.2, 208510_s_at
FIG. 1278: PRO84395
FIG. 1279A-C: DNA328614, SRRM2, 208610_s_at
FIG. 1280: PRO84396
FIG. 1281A-C: DNA328615, NP_003118.1,
208611_s_at
FIG. 1282: PRO84397
FIG. 1283A-C: DNA328616, NP_001448.1,
208613_s_at
FIG. 1284: PRO84398
FIG. 1285: DNA326362, VATI, 208626_s_at
FIG. 1286: PRO82758
FIG. 1287: DNA325912, NP_001093.1, 208637_x_at
FIG. 1288: PRO82367
FIG. 1289: DNA271268, NP_009057.1, 208649_s_at
FIG. 1290: PRO59579
FIG. 1291: DNA328617, AF299343, 208653_s_at
FIG. 1292: PRO84399
FIG. 1293A-C: DNA328618, NP_003307.2,
208664_s_at
FIG. 1294: PRO84400
FIG. 1295: DNA304686, NP_002565.1, 208680_at
FIG. 1296: PRO71112
FIG. 1297: DNA304499, NP_006588.1, 208687_x_at
FIG. 1298: PRO71063
FIG. 1299A-B: DNA328619, BC001188, 208691_at
FIG. 1300: PRO84401
FIG. 1301: DNA287189, NP_002038.1, 208693_s_at
FIG. 1302: PRO69475
FIG. 1303: DNA324217, ATIC, 208758_at
FIG. 1304: PRO80908
FIG. 1305: DNA327696, AF228339, 208763_s_at
FIG. 1306: PRO83679
FIG. 1307: DNA328620, AK000295, 208772_at
FIG. 1308: PRO84402
FIG. 1309: DNA328621, NP_002788.1, 208799_at
FIG. 1310: PRO84403
FIG. 1311: DNA287169, CAA42052.1, 2088051_at
FIG. 1312: PRO10404
FIG. 1313: DNA324531, NP_002120.1, 208808_s_at
FIG. 1314: PRO81185
FIG. 1315: DNA273521, NP_002070.1, 208813_at
FIG. 1316: PRO61502
FIG. 1317: DNA328622, BC000835, 208827_at
FIG. 1318: PRO82662
FIG. 1319: DNA227556, NP_001670.1, 208836_at
FIG. 1320: PRO38019
FIG. 1321: DNA326042, NP_031390.1, 208837_at
FIG. 1322: PRO1078
FIG. 1323A-B: DNA328623, NP_056107.1,
208858_s_at
FIG. 1324: PRO61321
FIG. 1325: DNA227874, NP_003320.1, 208864_s_at
FIG. 1326: PRO38337
FIG. 1327: DNA328624, BC003562, 208891_at
FIG. 1328: PRO59076
FIG. 1329: DNA328625, NP_073143.1, 208892_s_at
FIG. 1330: PRO84404
FIG. 1331: DNA328626, NP_057078.1, 208898_at
FIG. 1332: PRO61768
FIG. 1333: DNA327700, BC015130, 208905_at
FIG. 1334: PRO83683
FIG. 1335: DNA325472, NP_116056.2, 208906_at
FIG. 1336: PRO1995
FIG. 1337A-B: DNA328627, FLJ13052, 208918_s_at
FIG. 1338: PRO84405
FIG. 1339: DNA325473, NP_006353.2, 208922_s_at
FIG. 1340: PRO81996
FIG. 1341: DNA287238, NP_000425.1, 208926_at
FIG. 1342: PRO69515
FIG. 1343: DNA328628, NP_060542.2, 208933_s_at
FIG. 1344: PRO84406
FIG. 1345: DNA290261, NP_001291.2, 208960_s_at
FIG. 1346: PRO70387
FIG. 1347A-B: DNA325478, NP_037534.2,
208962_s_at
FIG. 1348: PRO81999
FIG. 1349: DNA328629, NP_006079.1, 208977_x_at
FIG. 1350: PRO84407
FIG. 1351: DNA328630, NP_036293.1, 209004_s_at
FIG. 1352: PRO84408
FIG. 1353: DNA328631, AK027318, 209006_s_at
FIG. 1354: PRO84409
FIG. 1355: DNA328632, DJ465N24.2.1Homo,
209007_s_at
FIG. 1356: DNA328633, NP_004784.2, 209017_s_at
FIG. 1357: PRO84411
FIG. 1358A-B: DNA328634, NP_006594.1,
209023_s_at
FIG. 1359: PRO84412
FIG. 1360: DNA328635, BC020946, 209026_x_at
FIG. 1361: PRO84413
FIG. 1362: DNA274202, NP_006804.1, 209034_at
FIG. 1363: PRO62131
FIG. 1364: DNA328636, PAPSS1, 209043_at
FIG. 1365: PRO84414
FIG. 1366A-C: DNA328637, HSA7042, 209053_s_at
FIG. 1367: PRO81109
FIG. 1368: DNA326406, NP_005315.1, 209069_s_at
FIG. 1369: PRO11403
FIG. 1370: DNA227289, NP_006532.1, 209080_x_at
FIG. 1371: PRO37752
FIG. 1372: DNA274180, NP_009005.1, 209083_at
FIG. 1373: PRO62110
FIG. 1374: DNA327707, NP_000148.1, 209093_s_at
FIG. 1375: PRO83689
FIG. 1376: DNA226564, NP_000099.1, 209095_at
FIG. 1377: PRO37027
FIG. 1378: DNA325163, NP_001113.1, 209122_at
FIG. 1379: PRO81730
FIG. 1380: DNA328638, BC000576, 209123_at
FIG. 1381: PRO81129
FIG. 1382: DNA274723, AAB62222.1, 209129_at
FIG. 1383: PRO62502
FIG. 1384: DNA328639, HSM801840, 209132_s_at
FIG. 1385: PRO84415
FIG. 1386: DNA328640, ASPH, 209135_at
FIG. 1387: PRO84416
FIG. 1388: DNA327713, BC010653, 209146_at
FIG. 1389: PRO37975
FIG. 1390: DNA271937, NP_055419.1, 209154_at
FIG. 1391: PRO60213
FIG. 1392: DNA328641, NP_001840.2, 209156_s_at
FIG. 1393: PRO84417
FIG. 1394: DNA325285, AKR1C3, 209160_at
FIG. 1395: PRO81832
FIG. 1396A-B: DNA328642, AF073310,
209184_s_at
FIG. 1397: PRO84418
FIG. 1398A-B: DNA328643, HUMHK1A,
209186_at
FIG. 1399: PRO84419
FIG. 1400: DNA189700, NP_005243.1, 209189_at
FIG. 1401: PRO25619
FIG. 1402: DNA327715, NP_115914.1, 209191_at
FIG. 1403: PRO83694
FIG. 1404: DNA103520, NP_002639.1, 209193_at
FIG. 1405: PRO4847
FIG. 1406A-B: DNA269816, MEF2C, 209199_s_at
FIG. 1407: PRO58219
FIG. 1408: DNA328644, 349746.9, 209200_at
FIG. 1409: PRO84420
FIG. 1410: DNA326891, NP_001748.1, 209213_at
FIG. 1411: PRO83212
FIG. 1412: DNA328645, NP_009006.1, 209216_at
FIG. 1413: PRO84421
FIG. 1414: DNA227483, NP_003120.1, 209218_at
FIG. 1415: PRO37946
FIG. 1416: DNA328646, NP_036517.1, 209230_s_at
FIG. 1417: PRO84422
FIG. 1418A-C: DNA328647, AB017133, 209234_at
FIG. 1419: PRO84423
FIG. 1420A-B: DNA328648, D87075, 209236_at
FIG. 1421: DNA328649, NP_116093.1, 20925_x_at
FIG. 1422: PRO84424
FIG. 1423: DNA255255, NP_071437.1, 209267_s_at
FIG. 1424: PRO50332
FIG. 1425A-B: DNA226827, NP_001673.1,
209281_s_at
FIG. 1426: PRO37290
FIG. 1427: DNA328650, 200118.10, 209286_at
FIG. 1428: PRO84425
FIG. 1429: DNA274883, NP_000058.1, 209301_at
FIG. 1430: PRO62628
FIG. 1431: DNA328651, AF087853, 209305_s_at
FIG. 1432: PRO82889
FIG. 1433: DNA327718, CASP4, 209310_s_at
FIG. 1434: PRO83697
FIG. 1435: DNA328652, NP_077298.1, 209321_s_at
FIG. 1436: PRO84426
FIG. 1437: DNA328653, AF063020, 209337_at
FIG. 1438: PRO84427
FIG. 1439: DNA328654, UAP1, 209340_at
FIG. 1440: PRO84428
FIG. 1441: DNA328655, 346677.3, 209341_s_at
FIG. 1442: PRO84429
FIG. 1443: DNA269630, NP_003281.1, 209344_at
FIG. 1444: PRO58042
FIG. 1445A-B: DNA328656, HSA303098,
209345_s_at
FIG. 1446: PRO84430
FIG. 1447A-B: DNA328657, NP_060895.1,
209346_s_at
FIG. 1448: PRO84431
FIG. 1449A-B: DNA328658, AF055376,
209348_s_at
FIG. 1450: PRO84432
FIG. 1451: DNA327719, NP_003704.2, 209355_s_at
FIG. 1452: PRO83698
FIG. 1453: DNA328659, ECM1, 209365_s_at
FIG. 1454: PRO84433
FIG. 1455: DNA225952, NP_001267.1, 209395_at
FIG. 1456: PRO36415
FIG. 1457: DNA275366, BC001851, 209444_at
FIG. 1458: PRO63036
FIG. 1459: DNA328660, NP_003675.2, 209467_s_at
FIG. 1460: PRO84434
FIG. 1461A-B: DNA328661, NP_006304.1,
209475_at
FIG. 1462: PRO84435
FIG. 1463: DNA328662, OSBPL1A, 209485_s_at
FIG. 1464: PRO84436
FIG. 1465: DNA324899, NP_002938.1, 209507_at
FIG. 1466: PRO81503
FIG. 1467: DNA274027, HSU38654, 209515_s_at
FIG. 1468: PRO61971
FIG. 1469: DNA328663, NP_057157.1, 209524_at
FIG. 1470: PRO36183
FIG. 1471A-C: DNA328664, NP_009131.1,
209534_x_at
FIG. 1472: PRO84437
FIG. 1473A-B: DNA328665, RGL, 209568_s_at
FIG. 1474: PRO84438
FIG. 1475: DNA328666, AF084943, 209585_s_at
FIG. 1476: PRO1917
FIG. 1477: DNA328667, S69189, 209600_s_at
FIG. 1478: PRO84439
FIG. 1479: DNA328668, NP_003157.1, 209607_x_at
FIG. 1480: PRO84440
FIG. 1481: DNA328669, NP_005882.1, 209608_s_at
FIG. 1482: PRO84441
FIG. 1483A-B: DNA328670, BC001618,
209610_s_at
FIG. 1484: PRO70011
FIG. 1485: DNA256209, NP_002259.1, 209653_at
FIG. 1486: PRO51256
FIG. 1487A-B: DNA272671, HSU26710, 209682_at
FIG. 1488: PRO60796
FIG. 1489: DNA151564, DNA151564, 209683_at
FIG. 1490: PRO11886
FIG. 1491: DNA327727, NP_000308.1, 209694_at
FIG. 1492: PRO83705
FIG. 1493: DNA328671, NP_000498.2, 209696_at
FIG. 1494: PRO84442
FIG. 1495: DNA327728, BC004492, 209703_x_at
FIG. 1496: PRO4348
FIG. 1497: DNA328672, CAA68871.1, 209707_at
FIG. 1498: PRO84444
FIG. 1499A-B: DNA328673, HUMCSDF1,
209716_at
FIG. 1500: PRO84445
FIG. 1501A-B: DNA304800, BC002538, 209723_at
FIG. 1502: PRO69458
FIG. 1503A-B: DNA328674, NP_056011.1,
209760_at
FIG. 1504: PRO84446
FIG. 1505: DNA324250, NP_536349.1, 209761_s_at
FIG. 1506: PRO80934
FIG. 1507A-B: DNA328675, ADAM19, 209765_at
FIG. 1508: PRO84447
FIG. 1509: DNA327731, NP_003302.1, 209803_s_at
FIG. 1510: PRO83707
FIG. 1511: DNA328676, IL16, 209827_s_at
FIG. 1512: PRO84448
FIG. 1513A-B: DNA196499, AB002384, 209829_at
FIG. 1514: PRO24988
FIG. 1515: DNA328677, AF060511, 209836_x_at
FIG. 1516: PRO84449
FIG. 1517: DNA324805, NP_008978.1, 209846_s_at
FIG. 1518: PRO81419
FIG. 1519: DNA273915, NP_036215.1, 209864_at
FIG. 1520: PRO61867
FIG. 1521: DNA290585, NP_000573.1, 209875_s_at
FIG. 1522: PRO70536
FIG. 1523: DNA328678, NP_008843.1, 209882_at
FIG. 1524: PRO62586
FIG. 1525: DNA328679, 347423.1, 209892_at
FIG. 1526: PRO84450
FIG. 1527: DNA328258, HSM802616, 209900_s_at
FIG. 1528: PRO84151
FIG. 1529A-B: DNA328680, NP_062541.1,
209907_s_at
FIG. 1530: PRO84451
FIG. 1531: DNA299884, AB040875, 209921_at
FIG. 1532: PRO70858
FIG. 1533: DNA328681, NP_005089.1, 209928_s_at
FIG. 1534: PRO84452
FIG. 1535: DNA272326, NP_006154.1, 209930_s_at
FIG. 1536: PRO60583
FIG. 1537: DNA328682, AF225981, 209935_at
FIG. 1538: PRO84453
FIG. 1539: DNA327754, NP_150634.1, 209970_x_at
FIG. 1540: PRO4526
FIG. 1541: DNA328683, NP_000399.1, 210007_s_at
FIG. 1542: PRO84454
FIG. 1543: DNA227660, NP_001327.1, 210042_s_at
FIG. 1544: PRO38123
FIG. 1545: DNA327739, AF092535, 210058_at
FIG. 1546: PRO83714
FIG. 1547: DNA327740, NP_003944.1, 210087_s_at
FIG. 1548: PRO1787
FIG. 1549: DNA328684, BC001234, 210102_at
FIG. 1550: PRO84455
FIG. 1551A-B: DNA328685, NP_127497.1,
210113_s_at
FIG. 1552: PRO34751
FIG. 1553: DNA328686, NP_000566.1, 210118_s_at
FIG. 1554: PRO64
FIG. 1555: DNA227757, NP_000743.1, 210128_s_at
FIG. 1556: PRO38220
FIG. 1557: DNA227501, NP_000295.1, 210139_s_at
FIG. 1558: PRO37964
FIG. 1559: DNA328687, AF004231, 210146_x_at
FIG. 1560: PRO84456
FIG. 1561A-B: DNA328688, NP_006838.2,
210152_at
FIG. 1562: PRO84457
FIG. 1563: DNA328689, NP_003259.2, 210166_at
FIG. 1564: PRO7521
FIG. 1565: DNA270196, HUMZFM1B, 210172_at
FIG. 1566: PRO58584
FIG. 1567: DNA328690, NP_524145.1, 210240_s_at
FIG. 1568: PRO59660
FIG. 1569: DNA326963, HRIHFB2122, 210276_s_at
FIG. 1570: PRO83276
FIG. 1571: DNA328691, NP_065717.1, 210346_s_at
FIG. 1572: PRO84458
FIG. 1573: DNA227652, NP_002549.1, 210401_at
FIG. 1574: PRO38115
FIG. 1575: DNA225514, NP_003864.1, 210510_s_at
FIG. 1576: PRO35977
FIG. 1577: DNA216517, NP_005055.1, 210549_s_at
FIG. 1578: PRO34269
FIG. 1579: DNA327746, HUMGCBA, 210589_s_at
FIG. 1580: PRO83720
FIG. 1581: DNA328692, AF025529, 210660_at
FIG. 1582: PRO84459
FIG. 1583: DNA272127, NP_003928.1, 210663_s_at
FIG. 1584: PRO60397
FIG. 1585: DNA326525, NP_006330.1, 210719_s_at
FIG. 1586: PRO82894
FIG. 1587: DNA226183, NP_001453.1, 210773_s_at
FIG. 1588: PRO36646
FIG. 1589: DNA226078, NP_000296.1, 210830_s_at
FIG. 1590: PRO36541
FIG. 1591: DNA226152, NP_002650.1, 210845_s_at
FIG. 1592: PRO36615
FIG. 1593: DNA328693, HSU03891, 210873_x_at
FIG. 1594: PRO84460
FIG. 1595: DNA328694, BC007810, 210944_s_at
FIG. 1596: PRO84461
FIG. 1597: DNA213676, NP_004604.1, 211003_x_at
FIG. 1598: PRO35142
FIG. 1599: DNA328695, NP_002145.1, 211015_s_at
FIG. 1600: PRO61480
FIG. 1601: DNA328696, NP_009214.1, 211026_s_at
FIG. 1602: PRO62720
FIG. 1603: DNA328697, NP_116112.1, 211038_s_at
FIG. 1604: PRO84462
FIG. 1605: DNA328698, BC006403, 211063_s_at
FIG. 1606: PRO12168
FIG. 1607: DNA326712, NP_001285.1, 211136_s_at
FIG. 1608: PRO83054
FIG. 1609A-B: DNA328699, AF189723,
211137_s_at
FIG. 1610: PRO84463
FIG. 1611: DNA327752, HSDHACTYL,
211150_s_at
FIG. 1612A-B: DNA328700, SCD, 211162_x_at
FIG. 1613: PRO84464
FIG. 1614: DNA328701, PSEN2, 211373_s_at
FIG. 1615: PRO80745
FIG. 1616: DNA328702, NP_036519.1, 211413_s_at
FIG. 1617: PRO84465
FIG. 1618: DNA256637, NP_008849.1, 211423_s_at
FIG. 1619: PRO51621
FIG. 1620: DNA328703, NP_003956.1, 211434_s_at
FIG. 1621: PRO1873
FIG. 1622: DNA327755, NP_115957.1, 211458_s_at
FIG. 1623: PRO83725
FIG. 1624A-B: DNA328704, FGFR1, 211535_s_at
FIG. 1625: PRO34231
FIG. 1626: DNA324626, RIL, 211564_s_at
FIG. 1627: PRO81272
FIG. 1628: DNA328705, NP_001345.1, 211653_x_at
FIG. 1629: PRO62617
FIG. 1630: DNA328706, BC021909, 211714_x_at
FIG. 1631: PRO10347
FIG. 1632A-B: DNA328707, AF172264,
211828_s_at
FIG. 1633: PRO84466
FIG. 1634: DNA226582, NP_003863.1, 211844_s_at
FIG. 1635: PRO37045
FIG. 1636: DNA151912, BAA06683.1, 211935_at
FIG. 1637: PRO12756
FIG. 1638: DNA325941, NP_005339.1, 211968_s_at
FIG. 1639: PRO82388
FIG. 1640: DNA287433, NP_006810.1, 212009_s_at
FIG. 1641: PRO69690
FIG. 1642: DNA328708, NP_002678.1, 212036_s_at
FIG. 1643: PRO84467
FIG. 1644: DNA103380, NP_003365.1, 212038_s_at
FIG. 1645: PRO4710
FIG. 1646: DNA328709, BC004151, 212048_s_at
FIG. 1647: PRO37676
FIG. 1648A-B: DNA254751, AB018353, 212074_at
FIG. 1649: DNA328710, HUMLAMA, 212086_x_at
FIG. 1650A-B: DNA298616, NP_001839.1,
212091_s_at
FIG. 1651: PRO71027
FIG. 1652: DNA154139, DNA154139, 212099_at
FIG. 1653: DNA328711, AK023154, 212115_at
FIG. 1654: PRO84468
FIG. 1655: DNA328712, NP_006501.1, 212118_at
FIG. 1656: PRO84469
FIG. 1657: DNA328713, AF100737, 212130_x_at
FIG. 1658: PRO84470
FIG. 1659: DNA328714, HSM801966, 212146_at
FIG. 1660A-B: DNA151915, BAA09764.1,
212149_at
FIG. 1661: PRO12758
FIG. 1662: DNA88630, AAA52701.1, 212154_at
FIG. 1663: PRO2877
FIG. 1664: DNA328715, BC000950, 212160_at
FIG. 1665: DNA328716, HSM800707, 212179_at
FIG. 1666A-C: DNA255018, CAB61363.1,
212207_at
FIG. 1667: PRO50107
FIG. 1668A-B: DNA328717, CAB70761.1,
212232_at
FIG. 1669: PRO84473
FIG. 1670: DNA196116, DNA196116, 212246_at
FIG. 1671A-B: DNA254262, NP_055197.1,
212255_s_at
FIG. 1672: PRO49373
FIG. 1673: DNA327771, NP_109591.1, 212268_at
FIG. 1674: PRO83737
FIG. 1675A-B: DNA328718, AAC39776.1,
212285_s_at
FIG. 1676: PRO84474
FIG. 1677: DNA328719, BC012895, 212295_s_at
FIG. 1678: PRO84475
FIG. 1679: DNA271103, NP_005796.1, 212296_at
FIG. 1680: PRO59425
FIG. 1681A-B: DNA328720, HSA306929,
212297_at
FIG. 1682: PRO84476
FIG. 1683A-B: DNA328721, 1450005.12, 212298_at
FIG. 1684: PRO84477
FIG. 1685A-B: DNA150464, BAA34466.1,
212311_at
FIG. 1686: PRO12270
FIG. 1687: DNA326808, BC019307, 212312_at
FIG. 1688: PRO83141
FIG. 1689A-B: DNA124122, NP_005602.2,
212332_at
FIG. 1690: PRO6323
FIG. 1691: DNA287190, CAB43217.1, 212333_at
FIG. 1692: PRO69476
FIG. 1693A-B: DNA255527, HUMTI227HC,
212337_at
FIG. 1694: DNA328722, BC012469, 212341_at
FIG. 1695: PRO84478
FIG. 1696: DNA328723, S47833, 212360_at
FIG. 1697: PRO36682
FIG. 1698A-B: DNA328724, AB007856, 212367_at
FIG. 1699A-B: DNA327773, BAA25456.1,
212368_at
FIG. 1700: PRO83739
FIG. 1701A-C: DNA328725, AB007923, 212390_at
FIG. 1702A-B: DNA150950, BAA07645.1,
212396_s_at
FIG. 1703: PRO12554
FIG. 1704A-B: DNA328726, BAA25466.2,
212443_at
FIG. 1705: PRO84480
FIG. 1706: DNA328727, AB033105, 212453_at
FIG. 1707A-B: DNA328728, 481567.2, 212458_at
FIG. 1708: PRO84482
FIG. 1709: DNA151348, DNA151348, 212463_at
FIG. 1710: PRO11726
FIG. 1711A-: DNA328729, D80001, 212486_s_at
FIG. 1712: PRO38526
FIG. 1713A-B: DNA328730, BAA74899.2,
212492_s_at
FIG. 1714: PRO84483
FIG. 1715A-B: DNA328731, 234169.5, 212500_at
FIG. 1716: PRO84484
FIG. 1717: DNA328732, NP_116193.1, 212502_at
FIG. 1718: PRO84485
FIG. 1719: DNA0, AF038183, 212527_at
FIG. 1720: PRO
FIG. 1721: DNA328734, AAH01171.1, 212539_at
FIG. 1722: PRO84487
FIG. 1723: DNA328735, PHIP, 212542_s_at
FIG. 1724: PRO84488
FIG. 1725: DNA328736, BC009846, 212552_at
FIG. 1726: PRO84489
FIG. 1727A-D: DNA328737, 148650.1, 212560_at
FIG. 1728: PRO84490
FIG. 1729: DNA270260, HSPDCE2, 212568_s_at
FIG. 1730A-B: DNA328738, BAA31625.1,
212569_at
FIG. 1731: PRO84491
FIG. 1732A-B: DNA328739, PTPRC, 212587_s_at
FIG. 1733: PRO84492
FIG. 1734: DNA327776, 1379302.1, 212593_s_at
FIG. 1735: PRO83742
FIG. 1736: DNA151487, DNA151487, 212594_at
FIG. 1737: PRO11833
FIG. 1738A-B: DNA328740, BAA76781.1,
212611_at
FIG. 1739: PRO84493
FIG. 1740: DNA81753, DNA81753, 212613_at
FIG. 1741: PRO9216
FIG. 1742A-B: DNA253817, BAA20767.1,
212615_at
FIG. 1743: PRO49220
FIG. 1744A-B: DNA328741, 474863.12, 212622_at
FIG. 1745: PRO84494
FIG. 1746: DNA194679, BAA05062.1, 212623_at
FIG. 1747: PRO23989
FIG. 1748A-B: DNA328742, 244522.6, 212628_at
FIG. 1749: PRO59047
FIG. 1750: DNA270683, NP_006247.1, 212629_s_at
FIG. 1751: PRO59047
FIG. 1752A-D: DNA327777, HSIL1RECA,
212657_s_at
FIG. 1753A-B: DNA150762, BAA13197.1,
212658_at
FIG. 1754: PRO12455
FIG. 1755: DNA327838, NP_000568.1, 212659_s_at
FIG. 1756: PRO83789
FIG. 1757: DNA328743, 1234685.2, 212667_at
FIG. 1758: PRO84495
FIG. 1759: DNA328744, AF318364, 212680_x_at
FIG. 1760: PRO84496
FIG. 1761: DNA328745, 482138.6, 212687_at
FIG. 1762: PRO84497
FIG. 1763: DNA324378, NP_000523.1, 212694_s_at
FIG. 1764: PRO81047
FIG. 1765: DNA328746, CAB43213.1, 212698_s_at
FIG. 1766: PRO84498
FIG. 1767A-B: DNA328747, BAA83030.1,
212765_at
FIG. 1768: PRO84499
FIG. 1769A-B: DNA328748, HSJ001388, 212774_at
FIG. 1770: PRO59570
FIG. 1771: DNA328749, HSM802266, 212779_at
FIG. 1772: DNA328750, 7689361.1, 212812_at
FIG. 1773: PRO84500
FIG. 1774A-B: DNA328751, AF012086,
212842_x_at
FIG. 1775: DNA328752, CAA76270.1, 212864_at
FIG. 1776: PRO84501
FIG. 1777A-B: DNA328753, BAA13212.1,
212873_at
FIG. 1778: PRO84502
FIG. 1779: DNA271630, DNA271630, 212907_at
FIG. 1780: DNA328754, 1397726.9, 212912_at
FIG. 1781: PRO84503
FIG. 1782A-B: DNA328755, BAA25490.1,
212946_at
FIG. 1783: PRO84504
FIG. 1784A-B: DNA328756, BAA74893.2,
212975_at
FIG. 1785: PRO84505
FIG. 1786: DNA154982, DNA154982, 213034_at
FIG. 1787: DNA327785, BC017336, 213061_s_at
FIG. 1788: PRO83749
FIG. 1789A-C: DNA328757, 475076.9, 213069_at
FIG. 1790: PRO84506
FIG. 1791A-B: DNA328758, AB011123, 213109_at
FIG. 1792: DNA272600, NP_057259.1, 213112_s_at
FIG. 1793: PRO60737
FIG. 1794: DNA326217, NP_004474.1, 213129_s_at
FIG. 1795: PRO82630
FIG. 1796: DNA228053, DNA228053, 213158_at
FIG. 1797A-G: DNA103535, AF027153, 213164_at
FIG. 1798: PRO4862
FIG. 1799: DNA150875, CAB45717.1, 213246_at
FIG. 1800: PRO11589
FIG. 1801: DNA328759, HUMLPACI09, 213258_at
FIG. 1802: DNA328760, 1376674.1, 213274_s_at
FIG. 1803: PRO84508
FIG. 1804A-B: DNA328761, BAA82991.1,
213280_at
FIG. 1805: PRO84509
FIG. 1806: DNA260974, NP_006065.1, 213293_s_at
FIG. 1807: PRO54720
FIG. 1808: DNA328762, AAL30845.1, 213338_at
FIG. 1809: PRO84510
FIG. 1810: DNA327789, 1449824.5, 213348_at
FIG. 1811: PRO83753
FIG. 1812: DNA328763, NP_001219.2, 213373_s_at
FIG. 1813: PRO84511
FIG. 1814: DNA328764, NP_438169.1, 213375_s_at
FIG. 1815: PRO84512
FIG. 1816: DNA328765, 411350.1, 213391_at
FIG. 1817: PRO84513
FIG. 1818: DNA106195, DNA106195, 213454_at
FIG. 1819: DNA327795, BC014226, 213457_at
FIG. 1820: DNA328766, NP_006077.1, 213476_x_at
FIG. 1821: PRO84514
FIG. 1822: DNA328767, BC008767, 213501_at
FIG. 1823: PRO84515
FIG. 1824: DNA254264, HSM800224, 213546_at
FIG. 1825: PRO49375
FIG. 1826: DNA328768, 1194561.1, 213572_s_at
FIG. 1827: PRO84516
FIG. 1828: DNA327800, 1251176.10, 213593_s_at
FIG. 1829: PRO83763
FIG. 1830: DNA151422, DNA151422, 213605_s_at
FIG. 1831: PRO11792
FIG. 1832: DNA225974, NP_000864.1, 213620_s_at
FIG. 1833: PRO36437
FIG. 1834: DNA328769, CAA69330.1, 213624_at
FIG. 1835: PRO84517
FIG. 1836: DNA260173, DNA260173, 213638_at
FIG. 1837: PRO54102
FIG. 1838A-C: DNA273792, DNA273792,
213649_at
FIG. 1839: DNA151886, CAB43234.1, 213682_at
FIG. 1840: PRO12745
FIG. 1841: DNA227788, NP_002995.1, 213716_s_at
FIG. 1842: PRO38251
FIG. 1843: DNA328771, HSMYOSIE, 213733_at
FIG. 1844: DNA328772, AAC19149.1, 213761_at
FIG. 1845: PRO84519
FIG. 1846: DNA328773, BC001528, 213766_x_at
FIG. 1847: PRO84520
FIG. 1848: DNA328774, NP_004263.1, 213793_s_at
FIG. 1849: PRO60536
FIG. 1850A-B: DNA328775, NP_006540.2,
213812_s_at
FIG. 1851: PRO84521
FIG. 1852: DNA328776, 407661.4, 213817_at
FIG. 1853: PRO84522
FIG. 1854A-B: DNA328777, IDN3, 213918_s_at
FIG. 1855: PRO84523
FIG. 1856: DNA196110, DNA196110, 214016_s_at
FIG. 1857: PRO24635
FIG. 1858: DNA150990, NP_003632.1, 214022_s_at
FIG. 1859: PRO12570
FIG. 1860: DNA328778, 234498.37, 214093_s_at
FIG. 1861: PRO84524
FIG. 1862A-B: DNA272292, NP_055459.1,
214130_s_at
FIG. 1863: PRO60550
FIG. 1864: DNA82378, NP_002695.1, 214146_s_at
FIG. 1865: PRO1725
FIG. 1866A-B: DNA328779, 332730.12,
214155_s_at
FIG. 1867: PRO84525
FIG. 1868: DNA304659, NP_002023.1, 214211_at
FIG. 1869: PRO71086
FIG. 1870: DNA256662, NP_009112.1, 214219_x_at
FIG. 1871: PRO51628
FIG. 1872A-B: DNA328780, 480940.15, 214285_at
FIG. 1873: PRO84526
FIG. 1874: DNA328781, 1453703.13, 214349_at
FIG. 1875: PRO84527
FIG. 1876: DNA273174, NP_001951.1, 214394_x_at
FIG. 1877: PRO61211
FIG. 1878: DNA328782, 337794.1, 214405_at
FIG. 1879: PRO84528
FIG. 1880: DNA287630, NP_000160.1, 214430_at
FIG. 1881: PRO2154
FIG. 1882: DNA227376, NP_005393.1, 214435_x_at
FIG. 1883: PRO37839
FIG. 1884: DNA273138, NP_005495.1, 214452_at
FIG. 1885: PRO61182
FIG. 1886: DNA327812, NP_006408.2, 214453_s_at
FIG. 1887: PRO83773
FIG. 1888: DNA302598, NP_066361.1, 214487_s_at
FIG. 1889: PRO62511
FIG. 1890: DNA328783, NP_002021.2, 214560_at
FIG. 1891: PRO84529
FIG. 1892: DNA324728, BC017730, 214581_x_at
FIG. 1893: PRO868
FIG. 1894A-B: DNA328784, 331045.1, 214582_at
FIG. 1895: PRO84530
FIG. 1896: DNA328785, NP_004062.1, 214683_s_at
FIG. 1897: PRO84531
FIG. 1898: DNA328786, BC017407, 214686_at
FIG. 1899: PRO84532
FIG. 1900: DNA271990, DNA271990, 214722_at
FIG. 1901A-B: DNA274485, AB007863, 214735_at
FIG. 1902: DNA328787, 238292.8, 214746_s_at
FIG. 1903: PRO84533
FIG. 1904: DNA328788, AK023937, 214763_at
FIG. 1905: PRO29183
FIG. 1906A-B: DNA328789, 344240.3, 214770_at
FIG. 1907: PRO84534
FIG. 1908A-B: DNA328790, 481415.9, 214786_at
FIG. 1909: PRO84535
FIG. 1910: DNA328791, 1383762.1, 214790_at
FIG. 1911: PRO84536
FIG. 1912: DNA328792, 7692351.10, 214830_at
FIG. 1913: PRO84537
FIG. 1914: DNA328314, BC022780, 214841_at
FIG. 1915: PRO84182
FIG. 1916: DNA83102, DNA83102, 214866_at
FIG. 1917: PRO2591
FIG. 1918: DNA161326, DNA161326, 214934_at
FIG. 1919: DNA328794, 1099353.2, 214974_x_at
FIG. 1920: PRO84539
FIG. 1921: DNA328795, AF057354, 214975_s_at
FIG. 1922: DNA328796, HSM800535, 215078_at
FIG. 1923: DNA328797, 000092.6, 215087_at
FIG. 1924: PRO84540
FIG. 1925: DNA328798, NP_002088.1, 215091_s_at
FIG. 1926: PRO84541
FIG. 1927: DNA328799, BC008376, 215101_s_at
FIG. 1928: PRO1721
FIG. 1929: DNA270522, NP_006013.1, 215111_s_at
FIG. 1930: PRO58899
FIG. 1931: DNA328800, 194537.1, 215224_at
FIG. 1932: PRO84542
FIG. 1933A-B: DNA327827, HSM800826,
215235_at
FIG. 1934A-B: DNA226905, NP_055672.1,
215342_s_at
FIG. 1935: PRO37368
FIG. 1936: DNA327831, NP_076956.1, 215380_s_at
FIG. 1937: PRO83783
FIG. 1938: DNA328801, 407831.1, 215392_at
FIG. 1939: PRO84543
FIG. 1940A-B: DNA328802, C6orf5, 215411_s_at
FIG. 1941: PRO84544
FIG. 1942: DNA275385, NP_002085.1, 215438_x_at
FIG. 1943: PRO63048
FIG. 1944: DNA328803, BAA91443.1, 215440_s_at
FIG. 1945: PRO84545
FIG. 1946: DNA328804, 403621.1, 215767_at
FIG. 1947: PRO84546
FIG. 1948A-B: DNA328805, BAA86482.1,
215785_s_at
FIG. 1949: PRO84547
FIG. 1950: DNA328806, 208045.1, 216109_at
FIG. 1951: PRO84548
FIG. 1952: DNA269532, NP_004802.1, 216250_s_at
FIG. 1953: PRO57948
FIG. 1954: DNA328807, AAH10129.1, 216483_s_at
FIG. 1955: PRO84549
FIG. 1956: DNA188349, NP_002973.1, 216598_s_at
FIG. 1957: PRO21884
FIG. 1958: DNA328808, 1099517.2, 216607_s_at
FIG. 1959: PRO84550
FIG. 1960: DNA328809, PTPN12, 216915_s_at
FIG. 1961: PRO4803
FIG. 1962: DNA328810, NP_001770.1, 216942_s_at
FIG. 1963: PRO2557
FIG. 1964A-C: DNA328811, NP_002213.1,
216944_s_at
FIG. 1965: PRO84551
FIG. 1966: DNA328812, BAA86575.1, 216997_x_at
FIG. 1967: PRO84552
FIG. 1968A-B: DNA328813, BAA76774.1,
217118_s_at
FIG. 1969: PRO84553
FIG. 1970A-B: DNA328814, HUMMHHLAJC,
217436_x_at
FIG. 1971A-B: DNA328815, 331104.2, 217521_at
FIG. 1972: PRO84554
FIG. 1973: DNA328816, 1446567.1, 217526_at
FIG. 1974: PRO84555
FIG. 1975A-B: DNA255619, AF054589,
217599_s_at
FIG. 1976: PRO50682
FIG. 1977: DNA327848, NP_005998.1, 217649_at
FIG. 1978: PRO83793
FIG. 1979: DNA328817, 1498470.1, 217678_at
FIG. 1980: PRO84556
FIG. 1981: DNA328818, NP_071435.1, 217730_at
FIG. 1982: PRO38175
FIG. 1983: DNA327935, NP_079422.1, 217745_s_at
FIG. 1984: PRO83866
FIG. 1985A-B: DNA88040, NP_000005.1, 217757_at
FIG. 1986: PRO2632
FIG. 1987A-B: DNA88226, NP_000055.1, 217767_at
FIG. 1988: PRO2237
FIG. 1989: DNA325821, NP_057016.1, 217769_s_at
FIG. 1990: PRO82287
FIG. 1991: DNA227358, NP_057479.1, 217777_s_at
FIG. 1992: PRO37821
FIG. 1993: DNA328819, NP_057145.1, 217781_s_at
FIG. 1994: PRO84557
FIG. 1995: DNA327850, NP_006546.1, 217785_s_at
FIG. 1996: PRO60803
FIG. 1997: DNA328303, NP_056525.1, 217807_s_at
FIG. 1998: PRO84173
FIG. 1999: DNA328820, NP_077022.1, 217808_s_at
FIG. 2000: PRO84558
FIG. 2001: DNA328821, NP_006708.1, 217813_s_at
FIG. 2002: PRO84559
FIG. 2003: DNA328822, AK001511, 217830_s_at
FIG. 2004: PRO84560
FIG. 2005: DNA328823, NP_057421.1, 217838_s_at
FIG. 2006: PRO84561
FIG. 2007: DNA226759, NP_054775.1, 217845_x_at
FIG. 2008: PRO37222
FIG. 2009: DNA327939, NP_060654.1, 217852_s_at
FIG. 2010: PRO83869
FIG. 2011A-B: DNA324921, NP_073585.6,
217853_at
FIG. 2012: PRO81523
FIG. 2013: DNA328824, DREV1, 217868_s_at
FIG. 2014: PRO84562
FIG. 2015: DNA225604, NP_057226.1, 217869_at
FIG. 2016: PRO36067
FIG. 2017: DNA326937, NP_002406.1, 217871_s_at
FIG. 2018: PRO83255
FIG. 2019: DNA255145, NP_060917.1, 217882_at
FIG. 2020: PRO50225
FIG. 2021A-B: DNA328825, 1398762.11, 217886_at
FIG. 2022: PRO84563
FIG. 2023: DNA189504, NP_064539.1, 217898_at
FIG. 2024: PRO25402
FIG. 2025: DNA328826, NP_004272.2, 217911_s_at
FIG. 2026: PRO84564
FIG. 2027: DNA328827, NP_076869.1, 217949_s_at
FIG. 2028: PRO21784
FIG. 2029: DNA328828, NP_067027.1, 217956_s_at
FIG. 2030: PRO84565
FIG. 2031: DNA328829, NP_057230.1, 217959_s_at
FIG. 2032: PRO84566
FIG. 2033: DNA328830, NP_061118.1, 217962_at
FIG. 2034: PRO84567
FIG. 2035: DNA327855, NP_057387.1, 217975_at
FIG. 2036: PRO83367
FIG. 2037: DNA328831, NP_057329.1, 217989_at
FIG. 2038: PRO233
FIG. 2039: DNA328832, NP_067022.1, 217995_at
FIG. 2040: PRO84568
FIG. 2041: DNA328833, BC018929, 217996_at
FIG. 2042: PRO84569
FIG. 2043: DNA328834, AF220656, 217997_at
FIG. 2044: DNA326005, NP_057004.1, 218007_s_at
FIG. 2045: PRO82446
FIG. 2046: DNA328835, NP_068760.1, 218019_s_at
FIG. 2047: PRO84571
FIG. 2048: DNA328836, NP_054894.1, 218027_at
FIG. 2049: PRO84572
FIG. 2050: DNA328837, NP_057149.1, 218046_s_at
FIG. 2051: PRO81876
FIG. 2052: DNA328838, NP_054797.2, 218049_s_at
FIG. 2053: PRO70319
FIG. 2054: DNA328839, NP_057180.1, 218059_at
FIG. 2055: PRO84573
FIG. 2056: DNA328840, NP_060481.1, 218067_s_at
FIG. 2057: PRO84574
FIG. 2058: DNA328841, NP_060557.2, 218073_s_at
FIG. 2059: PRO84575
FIG. 2060A-C: DNA328842, 235943.8, 218098_at
FIG. 2061: PRO84576
FIG. 2062: DNA328843, NP_060939.1, 218099_at
FIG. 2063: PRO84577
FIG. 2064: DNA328844, NP_061156.1, 218111_s_at
FIG. 2065: PRO82111
FIG. 2066: DNA227498, NP_002125.3, 218120_s_at
FIG. 2067: PRO37961
FIG. 2068: DNA328845, NP_060615.1, 218126_at
FIG. 2069: PRO10274
FIG. 2070: DNA227264, LOC51312, 218136_s_at
FIG. 2071: PRO37727
FIG. 2072: DNA327857, NP_057386.1, 218142_s_at
FIG. 2073: PRO83799
FIG. 2074: DNA325852, NP_078813.1, 218153_at
FIG. 2075: PRO82314
FIG. 2076: DNA328846, NP_060522.2, 218169_at
FIG. 2077: PRO84578
FIG. 2078: DNA228094, NP_079416.1, 218175_at
FIG. 2079: PRO38557
FIG. 2080: DNA328847, NP_056338.1, 218194_at
FIG. 2081: PRO84579
FIG. 2082: DNA150593, NP_054747.1, 218196_at
FIG. 2083: PRO12353
FIG. 2084: DNA256555, NP_060042.1, 218205_s_at
FIG. 2085: PRO51586
FIG. 2086: DNA328848, NP_004522.1, 218212_s_at
FIG. 2087: PRO84580
FIG. 2088: DNA271622, NP_006020.3, 218224_at
FIG. 2089: PRO59909
FIG. 2090: DNA324353, NP_004538.2, 218226_s_at
FIG. 2091: PRO81026
FIG. 2092: DNA328849, NP_057075.1, 218232_at
FIG. 2093: PRO4382
FIG. 2094: DNA328850, NP_057187.1, 218254_s_at
FIG. 2095: PRO84581
FIG. 2096: DNA273230, NP_060914.1, 218273_s_at
FIG. 2097: PRO61257
FIG. 2098: DNA328851, NP_068590.1, 218276_s_at
FIG. 2099: PRO84582
FIG. 2100: DNA323953, NP_003507.1, 218280_x_at
FIG. 2101: PRO80685
FIG. 2102: DNA254824, AF267865, 218294_s_at
FIG. 2103: PRO49920
FIG. 2104A-B: DNA328852, NP_003609.2,
218311_at
FIG. 2105: PRO84583
FIG. 2106A-B: DNA328853, NP_065702.2,
218319_at
FIG. 2107: PRO84584
FIG. 2108: DNA328854, NP_056979.1, 218350_s_at
FIG. 2109: PRO84585
FIG. 2110: DNA328855, NP_076952.1, 218375_at
FIG. 2111: PRO9771
FIG. 2112: DNA328856, NP_068376.1, 218380_at
FIG. 2113: PRO84586
FIG. 2114: DNA328857, NP_037481.1, 218407_x_at
FIG. 2115: PRO84587
FIG. 2116: DNA324953, NP_057412.1, 218412_s_at
FIG. 2117: PRO81550
FIG. 2118A-B: DNA255062, NP_060704.1,
218424_s_at
FIG. 2119: PRO50149
FIG. 2120: DNA150661, NP_057162.1, 218446_s_at
FIG. 2121: PRO12398
FIG. 2122: DNA326218, NP_064573.1, 218447_at
FIG. 2123: PRO82631
FIG. 2124: DNA328858, HEBP1, 218450_at
FIG. 2125: PRO84588
FIG. 2126: DNA327942, NP_060596.1, 218465_at
FIG. 2127: PRO83870
FIG. 2128: DNA328859, AF154054, 218468_s_at
FIG. 2129: PRO1608
FIG. 2130A-B: DNA328860, NP_037504.1,
218469_at
FIG. 2131: PRO1608
FIG. 2132: DNA328861, NP_057030.2, 218472_s_at
FIG. 2133: PRO84589
FIG. 2134: DNA328862, NP_057626.2, 218499_at
FIG. 2135: PRO84590
FIG. 2136: DNA328863, NP_060264.1, 218503_at
FIG. 2137: PRO84591
FIG. 2138: DNA328864, NP_060726.2, 218512_at
FIG. 2139: PRO84592
FIG. 2140: DNA255432, NP_060283.1, 218516_s_at
FIG. 2141: PRO50499
FIG. 2142: DNA194326, NP_065713.1, 218538_s_at
FIG. 2143: PRO23708
FIG. 2144: DNA328865, NP_064587.1, 218557_at
FIG. 2145: PRO84593
FIG. 2146: DNA328866, NP_005691.1, 218567_x_at
FIG. 2147: PRO69644
FIG. 2148: DNA328867, NP_085053.1, 218600_at
FIG. 2149: PRO84594
FIG. 2150: DNA328868, NP_057629.1, 218611_at
FIG. 2151: PRO84595
FIG. 2152: DNA328869, NP_060892.1, 218613_at
FIG. 2153: PRO84596
FIG. 2154: DNA328870, NP_060639.1, 218614_at
FIG. 2155: PRO84597
FIG. 2156: DNA256870, NP_073600.1, 218618_s_at
FIG. 2157: PRO51800
FIG. 2158: DNA254898, NP_060840.1, 218627_at
FIG. 2159: PRO49988
FIG. 2160: DNA328871, NP_068378.1, 218631_at
FIG. 2161: PRO84598
FIG. 2162: DNA328872, NP_036528.1, 218634_at
FIG. 2163: PRO84599
FIG. 2164: DNA328873, NP_057041.1, 218698_at
FIG. 2165: PRO84600
FIG. 2166: DNA324621, NP_054754.1, 218705_s_at
FIG. 2167: PRO1285
FIG. 2168: DNA328874, NP_054778.1, 218723_s_at
FIG. 2169: PRO84601
FIG. 2170: DNA328875, NP_064554.2, 218729_at
FIG. 2171: PRO84602
FIG. 2172: DNA328876, NP_060582.1, 218772_x_at
FIG. 2173: PRO84603
FIG. 2174: DNA328877, BC020507, 218821_at
FIG. 2175: PRO84604
FIG. 2176: DNA328878, NP_060104.1, 218823_s_at
FIG. 2177: PRO84605
FIG. 2178: DNA328879, NP_064570.1, 218845_at
FIG. 2179: PRO84606
FIG. 2180: DNA227367, NP_062456.1, 218853_s_at
FIG. 2181: PRO37830
FIG. 2182: DNA327872, NP_057713.1, 218856_at
FIG. 2183: PRO83812
FIG. 2184: DNA328880, NP_060369.1, 218872_at
FIG. 2185: PRO84607
FIG. 2186: DNA328881, NP_057706.1, 218890_x_at
FIG. 2187: PRO49469
FIG. 2188: DNA287166, NP_055129.1, 218943_s_at
FIG. 2189: PRO69459
FIG. 2190: DNA328882, NP_109589.1, 218967_s_at
FIG. 2191: PRO61822
FIG. 2192: DNA327211, NP_075053.1, 218989_x_at
FIG. 2193: PRO71052
FIG. 2194: DNA255929, NP_060935.1, 218992_at
FIG. 2195: PRO50982
FIG. 2196: DNA328883, NP_037474.1, 218996_at
FIG. 2197: PRO84608
FIG. 2198: DNA227194, FLJ11000, 218999_at
FIG. 2199: PRO37657
FIG. 2200: DNA328884, NP_054884.1, 219006_at
FIG. 2201: PRO84609
FIG. 2202: DNA227187, NP_057703.1, 219014_at
FIG. 2203: PRO37650
FIG. 2204: DNA328885, NP_061108.2, 219017_at
FIG. 2205: PRO50294
FIG. 2206A-B: DNA255239, NP_004832.1,
219026_s_at
FIG. 2207: PRO50316
FIG. 2208: DNA328886, NP_078811.1, 219040_at
FIG. 2209: PRO84610
FIG. 2210: DNA328887, NP_061907.1, 219045_at
FIG. 2211: PRO84611
FIG. 2212: DNA328888, NP_060436.1, 219053_s_at
FIG. 2213: PRO84612
FIG. 2214: DNA328889, NP_006005.1, 219061_s_at
FIG. 2215: PRO84613
FIG. 2216: DNA328890, NP_060403.1, 219093_at
FIG. 2217: PRO84614
FIG. 2218: DNA327877, NP_065108.1, 219099_at
FIG. 2219: PRO83816
FIG. 2220: DNA328891, NP_060263.1, 219143_s_at
FIG. 2221: PRO84615
FIG. 2222: DNA210216, NP_006860.1, 219150_s_at
FIG. 2223: PRO33752
FIG. 2224: DNA328892, NP_067643.2, 219165_at
FIG. 2225: PRO84616
FIG. 2226A-B: DNA328893, NP_065699.1,
219201_s_at
FIG. 2227: PRO9914
FIG. 2228: DNA287235, NP_060598.1, 219204_s_at
FIG. 2229: PRO69514
FIG. 2230: DNA225594, NP_037404.1, 219229_at
FIG. 2231: PRO36057
FIG. 2232: DNA328894, NP_060796.1, 219243_at
FIG. 2233: PRO84617
FIG. 2234: DNA328895, NP_071762.2, 219259_at
FIG. 2235: PRO1317
FIG. 2236: DNA328896, NP_079037.1, 219265_at
FIG. 2237: PRO84618
FIG. 2238: DNA328897, TRPV2, 219282_s_at
FIG. 2239: PRO12382
FIG. 2240: DNA273489, NP_055210.1, 219290_x_at
FIG. 2241: PRO61472
FIG. 2242A-B: DNA328898, NP_060261.1,
219316_s_at
FIG. 2243: PRO84619
FIG. 2244: DNA328899, NP_061024.1, 219326_s_at
FIG. 2245: PRO84620
FIG. 2246A-B: DNA255889, NP_061764.1,
219340_s_at
FIG. 2247: PRO50942
FIG. 2248: DNA328900, NP_060814.1, 219344_at
FIG. 2249: PRO84621
FIG. 2250: DNA254518, NP_057354.1, 219371_s_at
FIG. 2251: PRO49625
FIG. 2252: DNA188342, NP_064510.1, 219385_at
FIG. 2253: PRO21718
FIG. 2254: DNA256417, NP_077271.1, 219402_s_at
FIG. 2255: PRO51457
FIG. 2256A-B: DNA327887, NP_006656.1,
219403_s_at
FIG. 2257: PRO83823
FIG. 2258: DNA327888, NP_071732.1, 219412_at
FIG. 2259: PRO83824
FIG. 2260: DNA328901, FLJ20533, 219449_s_at
FIG. 2261: PRO84622
FIG. 2262: DNA328902, NP_071750.1, 219452_at
FIG. 2263: PRO84623
FIG. 2264: DNA328903, NP_002805.1, 219485_s_at
FIG. 2265: PRO84624
FIG. 2266: DNA328904, NP_076941.1, 219491_at
FIG. 2267: PRO84625
FIG. 2268A-B: DNA328905, NP_075392.1,
219496_at
FIG. 2269: PRO84626
FIG. 2270: DNA328906, NP_078855.1, 219506_at
FIG. 2271: PRO84627
FIG. 2272: DNA328907, NP_000067.1, 219534_x_at
FIG. 2273: PRO84628
FIG. 2274: DNA328908, 7691567.2, 219540_at
FIG. 2275: PRO84629
FIG. 2276: DNA225636, NP_065696.1, 219557_s_at
FIG. 2277: PRO36099
FIG. 2278A-B: DNA328909, NP_078800.2,
219558_at
FIG. 2279: PRO84630
FIG. 2280: DNA328910, NP_057666.1, 219593_at
FIG. 2281: PRO38848
FIG. 2282: DNA328911, MS4A4A, 219607_s_at
FIG. 2283: PRO84631
FIG. 2284: DNA328912, NP_060287.1, 219622_at
FIG. 2285: PRO84632
FIG. 2286: DNA328913, NP_079213.1, 219631_at
FIG. 2287: PRO84633
FIG. 2288: DNA328914, NP_060883.1, 219634_at
FIG. 2289: PRO36664
FIG. 2290: DNA327892, NP_060470.1, 219648_at
FIG. 2291: PRO83828
FIG. 2292: DNA328915, NP_055056.2, 219654_at
FIG. 2293: PRO84634
FIG. 2294: DNA228002, NP_071744.1, 219666_at
FIG. 2295: PRO38465
FIG. 2296: DNA328916, NP_071932.1, 219678_x_at
FIG. 2297: PRO84635
FIG. 2298: DNA287206, NP_060124.1, 219691_at
FIG. 2299: PRO69488
FIG. 2300: DNA328917, NP_061838.1, 219725_at
FIG. 2301: PRO7306
FIG. 2302: DNA328918, NP_078935.1, 219770_at
FIG. 2303: PRO84636
FIG. 2304: DNA328919, NP_078987.1, 219777_at
FIG. 2305: PRO84637
FIG. 2306: DNA227152, NP_038467.1, 219788_at
FIG. 2307: PRO37615
FIG. 2308: DNA328920, NP_061129.1, 219837_s_at
FIG. 2309: PRO4425
FIG. 2310: DNA256033, NP_060164.1, 219858_s_at
FIG. 2311: PRO51081
FIG. 2312: DNA254838, NP_078904.1, 219874_at
FIG. 2313: PRO49933
FIG. 2314: DNA328921, NP_057079.1, 219878_s_at
FIG. 2315: PRO84638
FIG. 2316: DNA256325, NP_005470.1, 219889_at
FIG. 2317: PRO51367
FIG. 2318: DNA328922, NP_037384.1, 219890_at
FIG. 2319: PRO84639
FIG. 2320: DNA328923, NP_075379.1, 219892_at
FIG. 2321: PRO84640
FIG. 2322: DNA256608, NP_060408.1, 219895_at
FIG. 2323: PRO51611
FIG. 2324: DNA328924, NP_057150.2, 219933_at
FIG. 2325: PRO84641
FIG. 2326: DNA255456, NP_057268.1, 219947_at
FIG. 2327: PRO50523
FIG. 2328: DNA227804, NP_065394.1, 219952_s_at
FIG. 2329: PRO38267
FIG. 2330: DNA328925, NP_076403.1, 220005_at
FIG. 2331: PRO84642
FIG. 2332: DNA256467, NP_079054.1, 220009_at
FIG. 2333: PRO51504
FIG. 2334A-B: DNA292946, NP_061160.1,
220023_at
FIG. 2335: PRO70613
FIG. 2336: DNA171414, NP_009130.1, 220034_at
FIG. 2337: PRO20142
FIG. 2338: DNA328926, NP_064703.1, 220046_s_at
FIG. 2339: PRO84643
FIG. 2340A-B: DNA221079, NP_071445.1,
220066_at
FIG. 2341: PRO34753
FIG. 2342: DNA256091, NP_071385.1, 220094_s_at
FIG. 2343: PRO51141
FIG. 2344: DNA328927, NP_078993.2, 220122_at
FIG. 2345: PRO84644
FIG. 2346: DNA328928, NP_068377.1, 220178_at
FIG. 2347: PRO84645
FIG. 2348: DNA324716, NP_463459.1, 220189_s_at
FIG. 2349: PRO81347
FIG. 2350: DNA228059, NP_073742.1, 220199_s_at
FIG. 2351: PRO38522
FIG. 2352: DNA328929, NP_060375.1, 220240_s_at
FIG. 2353: PRO84646
FIG. 2354A-B: DNA328930, NP_038465.1,
220253_s_at
FIG. 2355: PRO23525
FIG. 2356: DNA328931, NP_004226.1, 220266_s_at
FIG. 2357: PRO84647
FIG. 2358: DNA328932, NP_079057.1, 220301_at
FIG. 2359: PRO84648
FIG. 2360: DNA328933, NP_057466.1, 220307_at
FIG. 2361: PRO9891
FIG. 2362: DNA256735, NP_060175.1, 220333_at
FIG. 2363: PRO51669
FIG. 2364A-B: DNA328934, EML4, 220386_s_at
FIG. 2365: PRO84649
FIG. 2366: DNA328935, NP_009002.1, 220387_s_at
FIG. 2367: PRO84650
FIG. 2368: DNA254861, MCOLN3, 220484_at
FIG. 2369: PRO49953
FIG. 2370: DNA328936, NP_066998.1, 220491_at
FIG. 2371: PRO1003
FIG. 2372: DNA328937, PHEMX, 220558_x_at
FIG. 2373: PRO12380
FIG. 2374: DNA328938, NP_060617.1, 220643_s_at
FIG. 2375: PRO84651
FIG. 2376: DNA323756, NP_057267.2, 220688_s_at
FIG. 2377: PRO80512
FIG. 2378: DNA328939, NP_008834.1, 220741_s_at
FIG. 2379: PRO84652
FIG. 2380: DNA288247, NP_478059.1, 220892_s_at
FIG. 2381: PRO70011
FIG. 2382: DNA328940, NP_078893.1, 220933_s_at
FIG. 2383: PRO84653
FIG. 2384: DNA328941, NP_055218.2, 220937_s_at
FIG. 2385: PRO84654
FIG. 2386: DNA327953, NP_055182.2, 220942_x_at
FIG. 2387: PRO83878
FIG. 2388A-B: DNA323882, NP_000692.2,
220948_s_at
FIG. 2389: PRO80625
FIG. 2390: DNA327917, NP_112240.1, 220966_x_at
FIG. 2391: PRO83852
FIG. 2392: DNA328942, NP_112216.2, 220985_s_at
FIG. 2393: PRO84655
FIG. 2394: DNA328943, NP_036566.1, 221041_s_at
FIG. 2395: PRO51680
FIG. 2396: DNA328944, NP_060554.1, 221078_s_at
FIG. 2397: PRO84656
FIG. 2398: DNA328945, NP_079177.2, 221081_s_at
FIG. 2399: PRO84657
FIG. 2400: DNA328946, NP_055164.1, 221087_s_at
FIG. 2401: PRO12343
FIG. 2402: DNA328947, NP_055245.1, 221188_s_at
FIG. 2403: PRO84658
FIG. 2404: DNA257293, NP_110396.1, 221210_s_at
FIG. 2405: PRO51888
FIG. 2406: DNA327920, NP_110431.1, 221245_s_at
FIG. 2407: PRO83855
FIG. 2408A-C: DNA328287, NP_072174.2,
221246_x_at
FIG. 2409: PRO84163
FIG. 2410: DNA328948, NP_110437.1, 221253_s_at
FIG. 2411: PRO84659
FIG. 2412: DNA256432, NP_110415.1, 221266_s_at
FIG. 2413: PRO51471
FIG. 2414: DNA328027, NP_112570.2, 221437_s_at
FIG. 2415: PRO83944
FIG. 2416A-B: DNA272014, AF084555,
221482_s_at
FIG. 2417: PRO60289
FIG. 2418: DNA328949, AF157510, 221487_s_at
FIG. 2419: PRO84660
FIG. 2420: DNA328950, NP_057025.1, 221504_s_at
FIG. 2421: PRO84661
FIG. 2422A-B: DNA328951, HSM802232,
221523_s_at
FIG. 2423: PRO84662
FIG. 2424: DNA328952, NP_067067.1, 221524_s_at
FIG. 2425: PRO84663
FIG. 2426A-B: DNA273901, NP_110389.1,
221530_s_at
FIG. 2427: PRO61855
FIG. 2428: DNA274676, DKFZp564A176Homo,
221538_s_at
FIG. 2429: DNA328953, NP_004086.1, 221539_at
FIG. 2430: PRO70296
FIG. 2431A-B: DNA328954, NP_113664.1,
221541_at
FIG. 2432: PRO9851
FIG. 2433A-B: DNA269992, HUMACYLCOA,
221561_at
FIG. 2434: PRO58388
FIG. 2435: DNA328955, NP_054887.1, 221570_s_at
FIG. 2436: PRO84664
FIG. 2437A-B: DNA328956, AF110908, 221571_at
FIG. 2438: DNA188321, NP_004855.1, 221577_x_at
FIG. 2439: PRO21896
FIG. 2440: DNA328957, WBSCR5, 221581_s_at
FIG. 2441: PRO23859
FIG. 2442: DNA328958, BC001663, 221593_s_at
FIG. 2443: PRO84665
FIG. 2444: DNA328959, NP_077027.1, 221620_s_at
FIG. 2445: PRO4302
FIG. 2446: DNA254777, NP_055140.1, 221676_s_at
FIG. 2447: PRO49875
FIG. 2448: DNA327526, NP_065727.2, 221679_s_at
FIG. 2449: PRO83574
FIG. 2450: DNA328960, NP_076426.1, 221692_s_at
FIG. 2451: PRO84666
FIG. 2452: DNA327929, AK001785, 221748_s_at
FIG. 2453: PRO83861
FIG. 2454: DNA328961, NP_443112.1, 221756_at
FIG. 2455: PRO84667
FIG. 2456: DNA328962, BC021574, 221759_at
FIG. 2457: PRO82746
FIG. 2458A-B: DNA328963, 328765.9, 221760_at
FIG. 2459: PRO84668
FIG. 2460A-B: DNA327930, 1455324.9, 221765_at
FIG. 2461: PRO83862
FIG. 2462: DNA328964, AK056028, 221770_at
FIG. 2463: PRO84669
FIG. 2464A-C: DNA328965, AB051505, 221778_at
FIG. 2465A-B: DNA328966, BAB14908.1,
221790_s_at
FIG. 2466: PRO84670
FIG. 2467: DNA328967, BC017905, 221815_at
FIG. 2468: PRO84671
FIG. 2469: DNA274058, NP_057203.1, 221816_s_at
FIG. 2470: PRO61999
FIG. 2471A-B: DNA328968, 1322249.6, 221830_at
FIG. 2472: PRO62511
FIG. 2473: DNA272419, AF105036, 221841_s_at
FIG. 2474: PRO60672
FIG. 2475: DNA299882, DNA299882, 221872_at
FIG. 2476: PRO70856
FIG. 2477: DNA328969, 334394.2, 221878_at
FIG. 2478: PRO84672
FIG. 2479: DNA327933, 1452741.11, 221899_at
FIG. 2480: PRO83865
FIG. 2481: DNA328970, NP_057696.1, 221920_s_at
FIG. 2482: PRO84673
FIG. 2483: DNA328971, AK000472, 221923_s_at
FIG. 2484: PRO84674
FIG. 2485: DNA254787, AK023140, 221935_s_at
FIG. 2486: PRO49885
FIG. 2487: DNA327114, NP_006004.1, 221989_at
FIG. 2488: PRO62466
FIG. 2489: DNA328972, BC009950, 222001_x_at
FIG. 2490: DNA328973, NP_115538.1, 222024_s_at
FIG. 2491: PRO82497
FIG. 2492: DNA119482, DNA119482, 222108_at
FIG. 2493: PRO9850
FIG. 2494: DNA328974, NP_061893.1, 222116_s_at
FIG. 2495: PRO84676
FIG. 2496: DNA287209, NP_056350.1, 222154_s_at
FIG. 2497: PRO69490
FIG. 2498: DNA328975, NP_078807.1, 222155_s_at
FIG. 2499: PRO47688
FIG. 2500: DNA328976, BC019091, 222206_s_at
FIG. 2501: PRO84677
FIG. 2502: DNA256784, NP_075069.1, 222209_s_at
FIG. 2503: PRO51716
FIG. 2504: DNA328977, NP_071344.1, 222216_s_at
FIG. 2505: PRO84678
FIG. 2506: DNA328978, NP_060373.1, 222244_s_at
FIG. 2507: PRO84679
FIG. 2508A-B: DNA328979, 006242.19, 222266_at
FIG. 2509: PRO84680
FIG. 2510: DNA328980, 7692031.1, 222273_at
FIG. 2511: PRO84681
FIG. 2512: DNA328981, AF443871, 222294_s_at
FIG. 2513: PRO24633
FIG. 2514: DNA328982, 154391.1, 222313_at
FIG. 2515: PRO84682
FIG. 2516: DNA328983, 206335.1, 222366_at
FIG. 2517: PRO84683

### DETAILED DESCRIPTION OF THE PREFERRED EMBODIMENTS

I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO polypeptide” comprises a polypeptide having the same amino acid sequence as the corresponding PRO polypeptide derived from nature. Such native sequence PRO polypeptides can be isolated from nature or can be produced by recombinant or synthetic means. The term “native sequence PRO polypeptide” specifically encompasses naturally-occurring truncated or secreted forms of the specific PRO polypeptide (e.g., an extracellular domain sequence), naturally-occurring variant forms (e.g., alternatively spliced forms) and naturally-occurring allelic variants of the polypeptide. In various embodiments of the invention, the native sequence PRO polypeptides disclosed herein are mature or full-length native sequence polypeptides comprising the full-length amino acids sequences shown in the accompanying figures. Start and stop codons are shown in bold font and underlined in the figures. However, while the PRO polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO polypeptides.

The PRO polypeptide “extracellular domain” or “ECD” refers to a form of the PRO polypeptide which is essentially free of the transmembrane and cytoplasmic domains. Ordinarily, a PRO polypeptide ECD will have less than 1% of such transmembrane and/or cytoplasmic domains and preferably, will have less than 0.5% of such domains. It will be understood that any transmembrane domains identified for the PRO polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal-peptides” of the various PRO polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO polypeptide variant” means an active PRO polypeptide as defined above or below having at least about 80% amino acid sequence identity with a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Such PRO polypeptide variants include, for instance, PRO polypeptides wherein one or more amino acid residues are added, or deleted, at the N- or C-terminus of the full-length native amino acid sequence. Ordinarily, a PRO polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other specifically defined fragment of a full-length PRO polypeptide sequence as disclosed herein. Ordinarily, PRO variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the PRO polypeptide sequences identified herein is defined as the percentage of amino acid residues in a candidate sequence that are identical with the amino acid residues in the specific PRO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et. al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO variant polynucleotide” or “PRO variant nucleic acid sequence” means a nucleic acid molecule which encodes an active PRO polypeptide as defined below and which has at least about 80% nucleic acid sequence identity with a nucleotide acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Ordinarily, a PRO variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal sequence, as disclosed herein or any other fragment of a full-length PRO polypeptide sequence as disclosed herein. Variants do not encompass the native nucleotide sequence.

Ordinarily, PRO variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to PRO-encoding nucleic acid sequences identified herein is defined as the percentage of nucleotides in a candidate sequence that are identical with the nucleotides in the PRO nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the ark for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO variant polynucleotides are nucleic acid molecules that encode an active PRO polypeptide and which are capable of hybridizing, preferably under stringent hybridization and wash conditions, to nucleotide sequences encoding a full-length PRO polypeptide as disclosed herein. PRO variant polypeptides may be those that are encoded by a PRO variant polynucleotide.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO polypeptide natural environment will not be present. Ordinarily, however, isolated polypeptide will be prepared by at least one purification step.

An “isolated” PRO polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO monoclonal antibodies (including agonist, antagonist, and neutralizing antibodies), anti-PRO antibody compositions with polyepitopic specificity, single chain anti-PRO antibodies, and fragments of anti-PRO antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a PRO polypeptide which retain a biological and/or an immunological activity of native or naturally-occurring PRO, wherein “biological” activity refers to a biological function (either inhibitory or stimulatory) caused by a native or naturally-occurring PRO other than the ability to induce the production of an antibody against an antigenic epitope possessed by a native or naturally-occurring PRO and an “immunological” activity refers to the ability to induce the production of an antibody against an antigenic epitope possessed by a native or naturally-occurring PRO.

The term “antagonist” is used in the broadest sense, and includes any molecule that partially or fully blocks, inhibits, or neutralizes a biological activity of a native PRO polypeptide disclosed herein. In a similar manner, the term “agonist” is used in the broadest sense and includes any molecule that mimics a biological activity of a native PRO polypeptide disclosed herein. Suitable agonist or antagonist molecules specifically include agonist or antagonist antibodies or antibody fragments, fragments or amino acid sequence variants of native PRO polypeptides, peptides, antisense oligonucleotides, small organic molecules, etc. Methods for identifying agonists or antagonists of a PRO polypeptide may comprise contacting a PRO polypeptide with a candidate agonist or antagonist molecule and measuring a detectable change in one or more biological activities normally associated with the PRO polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments-originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies. vol. 113, Rosenburg and Moore eds., Springer-Verlag, New York, pp. 269-315(1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “monocyte/macrophage mediated disease” means a disease in which monocytes/macrophages directly or indirectly mediate or otherwise contribute to a morbidity in a mammal. The monocyte/macrophage mediated disease may be associated with cell mediated effects, lymphokine mediated effects, etc, and even effects associated with other immune cells if the cells are stimulated, for example, by the lymphokines secreted by monocytes/macrophages.

Examples of immune-related and inflammatory diseases, some of which are immune mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

The term “effective amount” is a concentration or amount of a PRO polypeptide and/or agonist/antagonist which results in achieving a particular stated purpose. An “effective amount” of a PRO polypeptide or agonist or antagonist thereof may be determined empirically. Furthermore, a “therapeutically effective amount” is a concentration or amount of a PRO polypeptide and/or agonist/antagonist which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor, prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor, transforming growth factors (TGFs) such as TGF-α and TGF-β; insulin-like growth factor-I and II; erythropoietin (EPO); osteoinductive factors, interferons such as interferon-α, -β, and -γ; colony stimulation factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison | NNNNLLLVV | (Length = 9 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Compositions and Methods of the Invention

A. Full-Length PRO Polypeptides

The present invention provides newly identified and isolated nucleotide sequences encoding polypeptides referred to in the present application as PRO polypeptides. In particular, cDNAs encoding various PRO polypeptides have been identified and isolated, as disclosed in further detail in the Examples below. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been disclosed. The predicted amino acid sequence can be determined from the nucleotide sequence using routine skill. For the PRO polypeptides and encoding nucleic acids described herein, Applicants have identified what is believed to be the reading frame best identifiable with the sequence information available at the time.

B. PRO Polypeptide Variants

In addition to the full-length native sequence PRO polypeptides described herein, it is contemplated that PRO variants can be prepared. PRO variants can be prepared by introducing appropriate nucleotide changes into the PRO DNA, and/or by synthesis of the desired PRO polypeptide. Those skilled in the art will appreciate that amino acid changes may alter post-translational processes of the PRO, such as changing the number or position of glycosylation sites or altering the membrane anchoring characteristics.

Variations in the native full-length sequence PRO or in various domains of the PRO described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO that results in a change in the amino acid sequence of the PRO as compared with the native sequence PRO. Optionally, the variation is by substitution of at least one amino acid with any other amino acid in one or more of the domains of the PRO. Guidance in determining which amino acid residue may be inserted, substituted or deleted without adversely affecting the desired activity may be found by comparing the sequence of the PRO with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO polypeptide.

PRO fragments may be prepared by any of a number of conventional techniques. Desired peptide fragments may be chemically synthesized. An alternative approach involves generating PRO fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO polypeptide fragments share at least one biological and/or immunological activity with the native PRO polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res. 13:4331 (1986); Zoller et al., Nucl. Acids Res. 10:6487 (1987)], cassette mutagenesis [Wells et al., Gene, 34:315 (1985)], restriction selection mutagenesis [Wells et al., Philos. Trans. R. Soc. London SerA, 317:415 (1986)]or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins. (W.H. Freeman & Co., N.Y.); Chothia, J. Mol. Biol. 150:1 (1976)]. If alanine substitution does not yield adequate amounts of variant, an isoteric amino acid can be used.

C. Modifications of PRO

Covalent modifications of PRO are included within the scope of this invention. One type of covalent modification includes reacting targeted amino acid residues of a PRO polypeptide with an organic derivatizing agent that is capable of reacting with selected side chains or the N- or C-terminal residues of the PRO. Derivatization with bifunctional agents is useful, for instance, for crosslinking PRO to a water-insoluble support matrix or surface for use in the method for purifying anti-PRO antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO polypeptide included within the scope of this invention comprises altering the native glycosylation pattern of the polypeptide. “Altering the native glycosylation pattern” is intended for purposes herein to mean deleting one or more carbohydrate moieties found in native sequence PRO (either by removing the underlying glycosylation site or by deleting the glycosylation by chemical and/or enzymatic means), and/or adding one or more glycosylation sites that are not present in the native sequence PRO. In addition, the phrase includes qualitative changes in the glycosylation of the native proteins, involving a change in the nature and proportions of the various carbohydrate moieties present.

Addition of glycosylation sites to the PRO polypeptide may be accomplished by altering the amino acid sequence. The alteration may be made, for example, by the addition of, or substitution by, one or more serine or threonine residues to the native sequence PRO (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem. 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO comprises linking the PRO polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

The PRO of the present invention may also be modified in a way to form a chimeric molecule comprising PRO fused to another, heterologous polypeptide or amino acid sequence.

In one embodiment, such a chimeric molecule comprises a fusion of the PRO with a tag polypeptide which provides an epitope to which an anti-tag antibody can selectively bind. The epitope tag is generally placed at the amino- or carboxyl-terminus of the PRO. The presence of such epitope-tagged forms of the PRO can be detected using an antibody against the tag polypeptide. Also, provision of the epitope tag enables the PRO to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6B10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6: 1204-1210 (1988)]; the KT3 epitope peptide [Martin et al., Science. 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO

The description below relates primarily to production of PRO by culturing cells transformed or transfected with a vector containing PRO nucleic acid. It is, of course, contemplated that alternative methods, which are well known in the art, may be employed to prepare PRO. For instance, the PRO sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc. 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO may be chemically synthesized separately and combined using chemical or enzymatic methods to produce the full-length PRO.

1. Isolation of DNA Encoding PRO

DNA encoding PRO may be obtained from a cDNA library prepared from tissue believed to possess the PRO mRNA and to express it at a detectable level. Accordingly, human PRO DNA can be conveniently obtained from a cDNA library prepared from human tissue, such as described in the Examples. The PRO-encoding gene may also be obtained from a genomic library or by known synthetic procedures (e.g., automated nucleic acid synthesis).

Libraries can be screened with probes (such as antibodies to the PRO or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra: Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology. 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r} ; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology 8:135 (1990)), K. thermotolerans, and K. marxianus; yarrowia (EP 402,226); Pichia pastoris (EP 183,070; Sreekishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biphys. Res. Commun., 112:284-289 (1983); Tilburn et al., Gene. 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene-encoding D-alanine racemase for Bacilli.

An example of suitable selectable markers for mammalian cells are those that enable the identification of cells competent to take up the PRO-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene. 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics 85:12 (1977)].

Expression and cloning vectors usually contain a promoter operably linked to the PRO-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO by higher eukaryotes may be increased by inserting an enhancer sequence into the vector. Enhancers are cis-acting elements of DNA, usually about from 10 to 300 bp, that act on a promoter to increase its transcription. Many sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO.

Still other methods, vectors, and host cells suitable for adaptation to the synthesis of PRO in recombinant vertebrate cell culture are described in Gething et al., Nature. 293:620-625 (1981); Mantei et al., Nature. 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA. 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO polypeptide or against a synthetic peptide based on the DNA sequences provided herein or against exogenous sequence fused to PRO DNA and encoding a specific antibody epitope.

5. Purification of Polypeptide

Forms of PRO may be recovered from culture medium or from host cell lysates. If membrane-bound, it can be released from the membrane using a suitable detergent solution (e.g. Triton-X 100) or by enzymatic cleavage. Cells employed in expression of PRO can be disrupted by various physical or chemical means, such as freeze-thaw cycling, sonication, mechanical disruption, or cell lysing agents.

It may be desired toto purify PRO from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO produced.

E. Tissue Distribution

The location of tissues expressing the PRO can be identified by determining mRNA expression in various human tissues. The location of such genes provides information about which tissues are most likely to be affected by the stimulating and inhibiting activities of the PRO polypeptides. The location of a gene in a specific tissue also provides sample tissue for the activity blocking assays discussed below.

As noted before, gene expression in various tissues may be measured by conventional Southern blotting, Northern blotting to quantitate the transcription of mRNA (Thomas, Proc. Natl. Acad. Sci. USA, 77:5201-5205 [1980]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes.

Gene expression in various tissues, alternatively, may be measured by immunological methods, such as immunohistochemical staining of tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence of a PRO polypeptide or against a synthetic peptide based on the DNA sequences encoding the PRO polypeptide or against an exogenous sequence fused to a DNA encoding a PRO polypeptide and encoding a specific antibody epitope. General techniques for generating antibodies, and special protocols for Northern blotting and in situ hybridization are provided below.

F. Antibody Binding Studies

The activity of the PRO polypeptides can be further verified by antibody binding studies, in which the ability of anti-PRO antibodies to inhibit the effect of the PRO polypeptides, respectively, on tissue cells is tested. Exemplary antibodies include polyclonal, monoclonal, humanized, bispecific, and heteroconjugate antibodies, the preparation of which will be described hereinbelow.

Antibody binding studies may be carried out in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tissue sample may be fresh or frozen or may be embedded in paraffin and fixed with a preservative such as formalin, for example.

G. Cell-Based Assays

Cell-based assays and animal models for immune related diseases can be used to further understand the relationship between the genes and polypeptides identified herein and the development and pathogenesis of immune related disease.

In a different approach, cells of a cell type known to be involved in a particular immune related disease are transfected with the cDNAs described herein, and the ability of these cDNAs to stimulate or inhibit immune function is analyzed. Suitable cells can be transfected with the desired gene, and monitored for immune function activity. Such transfected cell lines can then be used to test the ability of poly- or monoclonal antibodies or antibody compositions to inhibit or stimulate immune function, for example to modulate monocyte/macrophage proliferation or inflammatory cell infiltration. Cells transfected with the coding sequences of the genes identified herein can further be used to identify drug candidates for the treatment of immune related diseases.

In addition, primary cultures derived from transgenic animals (as described below) can be used in the cell-based assays herein, although stable cell lines are preferred. Techniques to derive continuous cell lines from transgenic animals are well known in the art (see, e.g., Small et al., Mol. Cell. Biol. 5: 642-648 [1985]).

The use of an agonist stimulating compound has also been validated experimentally. Activation of 4-1BB by treatment with an agonist anti-4-1BB antibody enhances eradication of tumors. Hellstrom, I. and Hellstrom, K. E., Crit. Rev. Immunol. (1998)18:1. Immunoadjuvant therapy for treatment of tumors, described in more detail below, is another example of the use of the stimulating compounds of the invention.

Alternatively, an immune stimulating or enhancing effect can also be achieved of a PRO which has vascular permeability enhancing properties. Enhanced vascular permeability would be beneficial to disorders which can be attenuated by local infiltration of immune cells (e.g., monocytes/macrophages, eosinophils, PMNs) and inflammation.

On the other hand, PRO polypeptides, as well as other compounds of the invention, which are direct inhibitors of monocyte/macrophage proliferation/activation, lymphokine secretion, and/or vascular permeability can be directly used to suppress the immune response. These compounds are useful to reduce the degree of the immune response and to treat immune related diseases characterized by a hyperactive, superoptimal, or autoimmune response. The use of compound which suppress vascular permeability would be expected to reduce inflammation. Such uses would be beneficial in treating conditions associated with excessive inflammation.

Alternatively, compounds, e.g., antibodies, which bind to stimulating PRO polypeptides and block the stimulating effect of these molecules produce a net inhibitory effect and can be used to suppress the monocyte/macrophage mediated immune response by inhibiting monocyte/macrophage proliferation/activation and/or lymphokine secretion. Blocking the stimulating effect of the polypeptides suppresses the immune response of the mammal.

H. Animal Models

The results of the cell based in vitro assays can be further verified using in vivo animal models and assays for monocyte/macrophage function. A variety of well known animal models can be used to further understand the role of the genes identified herein in the development and pathogenesis of immune related disease, and to test the efficacy of candidate therapeutic agents, including antibodies, and other antagonists of the native polypeptides, including small molecule antagonists. The in vivo nature of such models makes them predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, implantation under the renal capsule, etc.

Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing monocyte/macrophage reactivity against MHC antigens and minor transplant antigens. A suitable procedure is described in detail in Current Protocols in Immunology, above, unit 4.3.

Animal models for delayed type hypersensitivity provides an assay of cell mediated immune function as well. In chronic Delayed type hypersensitivity (DTH) reactions, monocytes that have differentiated into macrophages lead to the destruction of host tissue which is replaced by fibrous tissue (fibrosis).

Contact hypersensitivity is a simple delayed type hypersensitivity in vivo assay of cell mediated immune function. In this procedure, cutaneous exposure to exogenous haptens which gives rise to a delayed type hypersensitivity reaction which is measured and quantitated. Contact sensitivity involves an initial sensitizing phase followed by an elicitation phase. The elicitation phase occurs when the T lymphocytes encounter an antigen to which they have had previous contact. Swelling and inflammation occur, making this an excellent model of human allergic contact dermatitis. At this point, monocytes leave the blood and differentiate in to macrophages. A suitable procedure is described in detail in Current Protocols in Immunology, Eds. J. E. Cologan, A. M. Kruisbeek, D. H. Margulies, E. M. Shevach and W. Strober, John Wiley & Sons, Inc., 1994, unit 4.2. See also Grabbe, S. and Schwarz, T, Immun. Today 19 (1): 37-44 (1998)

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA 82, 6148-615 [1985]); gene targeting in embryonic stem cells (Thompson et al., Cell 56, 313-321 [1989]); electroporation of embryos (Lo, Mol. Cel. Biol. 3, 1803-1814 [1983]); sperm-mediated gene transfer (Lavitrano et al., Cell 57 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89, 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry.

The animals may be further examined for signs of immune disease pathology, for example by histological examination to determine infiltration of immune cells into specific tissues. Blocking experiments can also be performed in which the transgenic animals are treated with the compounds of the invention to determine the extent of the monocytes/macrophage proliferation stimulation or inhibition of the compounds. In these experiments, blocking antibodies which bind to the PRO polypeptide, prepared as described above, are administered to the animal and the effect on immune function is determined.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

I. ImmunoAdjuvant Therapy

In one embodiment, the immunostimulating compounds of the invention can be used in immunoadjuvant therapy for the treatment of tumors (cancer). It is now well established that monocytes/macrophages recognize human tumor specific antigens. One group of tumor antigens, encoded by the MAGE, BAGE and GAGE families of genes, are silent in all adult normal tissues, but are expressed in significant amounts in tumors, such as melanomas, lung tumors, head and neck tumors, and bladder carcinomas. DeSmet, C. et al., (1996) Proc. Natl. Acad. Sci. USA, 93:7149. It has been shown that stimulation of immune cells induces tumor regression and an antitumor response both in vitro and in vivo. Melero, I. et al., Nature Medicine (1997) 3:682; Kwon, E. D. et al., Proc. Natl. Acad. Sci. USA (1997) 94: 8099; Lynch, D. H. et al, Nature Medicine (1997) 3:625; Finn, O. J. and Lotze, M. T., J. Immunol. (1998) 21:114. The stimulatory compounds of the invention can be administered as adjuvants, alone or together with a growth regulating agent, cytotoxic agent or chemotherapeutic agent, to stimulate monocyte/macrophage proliferation/activation and an antitumor response to tumor antigens. The growth regulating, cytotoxic, or chemotherapeutic agent may be administered in conventional amounts using known administration regimes. Immunostimulating activity by the compounds of the invention allows reduced amounts of the growth regulating, cytotoxic, or chemotherapeutic agents thereby potentially lowering the toxicity to the patient.

J. Screening Assays for Drug Candidates

Screening assays for drug candidates are designed to identify compounds that bind to or complex with the polypeptides encoded by the genes identified herein or a biologically active fragment thereof, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art. All assays are common in that they call for contacting the drug candidate with a polypeptide encoded by a nucleic acid identified herein under conditions and for a time sufficient to allow these two components to interact.

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the candidate compound interacts with but does not bind to a particular protein encoded by a gene identified herein, its interaction with that protein can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London) 340, 245-246 (1989); Chien et al., Proc. Natl. Acad. Sci. USA 88, 9578-9582 (1991)] as disclosed by Chevray and Nathans, Proc. Natl. Acad. Sci. USA 89, 5789-5793 (1991). Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein protein-interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In order to find compounds that interfere with the interaction of a gene identified herein and other intra- or extracellular components can be tested, a reaction mixture is usually prepared containing the product of the gene and the intra- or extracellular component under conditions and for a time allowing for the interaction and binding of the two products. To test the ability of a test compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described above. The formation of a complex in the control reaction(s) but not in the reaction mixture containing the test compound indicates that the test compound interferes with the interaction of the test compound and its reaction partner.

K. Compositions and Methods for the Treatment of Immune Related Diseases

The compositions useful in the treatment of immune related diseases include, without limitation, proteins, antibodies, small organic molecules, peptides, phosphopeptides, antisense and ribozyme molecules, triple helix molecules, etc. that inhibit or stimulate immune function, for example, monocyte proliferation/activation, lymphokine release, or immune cell infiltration.

For example, antisense RNA and RNA molecules act to directly block the translation of mRNA by hybridizing to targeted mRNA and preventing protein translation. When antisense DNA is used, oligodeoxyribonucleotides derived from the translation initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4,469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed above and/or by any other screening techniques well known for those skilled in the art.

L. Anti-PRO Antibodies

The present invention further provides anti-PRO antibodies. Exemplary antibodies include polyclonal, monoclonal, humanized, bispecific, and heteroconjugate antibodies.

1. Polyclonal Antibodies

The anti-PRO antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-PRO antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-PRO antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature. 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature. 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95(1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

4. Bispecific Antibodies

Bispecific antibodies are monoclonal, preferably human or humanized, antibodies that have binding specificities for at least two different antigens. In the present case, one of the binding specificities is for the PRO, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature. 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast rumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies may bind to two different epitopes on a given PRO polypeptide herein. Alternatively, an anti-PRO polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PRO polypeptide. Bispecific antibodies may also be used to localize cytotoxic agents to cells which express a particular PRO polypeptide. These antibodies possess a PRO-binding arm and an arm which binds a cytotoxic agent or a radionuclide chelator, such as EOTUBE, DPTA, DOTA, or TETA. Another bispecific antibody of interest binds the PRO polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research. 53: 2560-2565 (1993). Alternatively, an antibody can be engineered that has dual Fc regions and may thereby have enhanced complement lysis and ADCC capabilities. See Stevenson et al., Anti-Cancer Drug Design. 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diptheria toxin, exotoxin A chain (from Pseudomonas aeruginosa) ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis(F-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a ‘ligand’ (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA. 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylemanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

M. Pharmaceutical Compositions

The active PRO molecules of the invention (e.g., PRO polypeptides, anti-PRO antibodies, and/or variants of each) as well as other molecules identified by the screening assays disclosed above, can be administered for the treatment of immune related diseases, in the form of pharmaceutical compositions.

Therapeutic formulations of the active PRO molecule, preferably a polypeptide or antibody of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Compounds identified by the screening assays disclosed herein can be formulated in an analogous manner, using standard techniques well known in the art.

Lipofections or liposomes can also be used to deliver the PRO molecule into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90, 7889-7893 [1993]).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active PRO molecules may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations or the PRO molecules may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

N. Methods of Treatment

It is contemplated that the polypeptides, antibodies and other active compounds of the present invention may be used to treat various immune related diseases and conditions, such as monocyte/macrophage diseases, including those characterized by infiltration of inflammatory cells into a tissue, stimulation of monocyte/macrophages, inhibition of monocytes/macrophages, increased or decreased vascular permeability or the inhibition thereof.

Exemplary conditions or disorders to be treated with the polypeptides, antibodies and other compounds of the invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes/macrophages into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestinal pneumonitis with pulmonary fibrosis, keratoconjunctivitis sicca, and rheumatoid nodules. The number and activation state of macrophages in the inflamed synovius correlates with the significance of RA (Kinne et al., 2000 Arthritis Res. 2: 189-202). As described above, macrophages are not believed to be involved in the early events of RA, but monocytes/macrophages have tissue destructive and tissue remodeling properties which may contribute to both acute and chronic RA.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. It was shown that CD163+ macrophages were increased in the synovial lining and colonic mucosa in Spondyloarthropathy and correlates with the expression of HLA-DR and the production of TNF-alpha (Baeten et al., 2002 J Pathol 196(3):343-350).

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. As well as T cells, monocytes/macrophages are proposed to play a role in the progression of scleroderma by secreting fibrogenic cytokines (Yamamoto et al., 2001 J Dermatol Sci 26(2): 133-139). Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjögren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitis can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Psoriasis is a T lymphocyte-mediated inflammatory disease. Lesions contain infiltrates of T lymphocytes, macrophages and antigen processing cells, and some neutrophils.

Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections. Molecules (or derivatives/agonists) which stimulate the immune reaction can be utilized therapeutically to enhance the immune response to infectious agents), diseases of immunodeficiency (molecules/derivatives/agonists) which stimulate the immune reaction can be utilized therapeutically to enhance the immune response for conditions of inherited, acquired, infectious induced (as in HIV infection), or iatrogenic (i.e., as from chemotherapy) immunodeficiency, and neoplasia.

It has been demonstrated that some human cancer patients develop an antibody and/or monocyte/macrophage response to antigens on neoplastic cells. It has also been shown in animal models of neoplasia that enhancement of the immune response can result in rejection or regression of that particular neoplasm. Molecules that enhance the monocyte/macrophage response have utility in vivo in enhancing the immune response against neoplasia. Molecules which enhance the monocyte/macrophage proliferative response (or small molecule agonists or antibodies that affected the same receptor in an agonistic fashion) can be used therapeutically to treat cancer. Molecules that inhibit the monocyte/macrophage response also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

The compounds of the present invention, e.g., polypeptides or antibodies, are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides and antibodies is preferred.

In immunoadjuvant therapy, other therapeutic regimens, such administration of an anti-cancer agent, may be combined with the administration of the proteins, antibodies or compounds of the instant invention. For example, the patient to be treated with a the immunoadjuvant of the invention may also receive an anti-cancer agent (chemotherapeutic agent) or radiation therapy. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the immunoadjuvant or may be given simultaneously therewith. Additionally, an anti-estrogen compound such as tamoxifen or an anti-progesterone such as onapristone (see, EP 616812) may be given in dosages known for such molecules.

It may be desirable to also administer antibodies against other immune disease associated or tumor associated antigens, such as antibodies which bind to CD20, CD11a, CD18, ErbB2, EGFR, ErbB3, ErbB4, or vascular endothelial factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the PRO polypeptides are coadministered with a growth inhibitory agent. For example, the growth inhibitory agent may be administered first, followed by a PRO polypeptide. However, simultaneous administration or administration first is also contemplated. Suitable dosages for the growth inhibitory agent are those presently used and may be lowered due to the combined action (synergy) of the growth inhibitory agent and the PRO polypeptide.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the attending physician. The compound is suitably administered to the patient at one time or over a series of treatments.

For example, depending on the type and severity of the disease, about 1 μg/kg to 15 mg/kg (e.g., 0.1-20 mg/kg) of polypeptide or antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

O. Articles of Manufacture

In another embodiment of the invention, an article of manufacture containing materials (e.g., comprising a PRO molecule) useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide or an antibody of the invention. An instruction or label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

P. Diagnosis and Prognosis of Immune Related Disease

Cell surface proteins, such as proteins which are overexpressed in certain immune related diseases, are excellent targets for drug candidates or disease treatment. The same proteins along with secreted proteins encoded by the genes amplified in immune related disease states find additional use in the diagnosis and prognosis of these diseases. For example, antibodies directed against the protein products of genes amplified in multiple sclerosis, rheumatoid arthritis, or another immune related disease, can be used as diagnostics or prognostics.

For example, antibodies, including antibody fragments, can be used to qualitatively or quantitatively detect the expression of proteins encoded by amplified or overexpressed genes (“marker gene products”). The antibody preferably is equipped with a detectable, e.g., fluorescent label, and binding can be monitored by light microscopy, flow cytometry, fluorimetry, or other techniques known in the art. These techniques are particularly suitable, if the overexpressed gene encodes a cell surface protein Such binding assays are performed essentially as described above.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Microarray Analysis of Monocyte/Macrophages

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (in this instance, differentiated macrophages) sample is greater than hybridization signal of a probe from a control (in this instance, non-differentiated monocytes) sample, the gene or genes expressed in the test tissue are identified. The implication of this result is that an overexpressed protein in a test tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

In this experiment, CD14+ monocytes are selected by positive selection according to Miltenyi MACS™ protocol. Lymphocytes in 100 ml heparinized blood are separated using Ficoll Paque™. Cells are washed twice in PBS/0.5% BSA/2 mM EDTA. In final wash, all gradients are pooled and volume is brought to approximately 10 ml. The cells are centrifuged, the supernatant is removed and the cell pellet is resuspended in buffer in a total volume of 10e7 cells per 80 μl buffer. Add 20 μl CD14 microbeds per 10e7 total cells, mix and incubate 15 minutes at 6-12 C. Wash the cells by adding 20× labeling volume of buffer, spin pellet and resuspend in 500 ul buffer per 10e8 cells. Separate cells with MACS™ depletion column type D and check purity of cells by labeling with anti-CD45 and anti-CD14 antibodies (cell purity at this point is >95%). Lyse cells in RNA lysis buffer to obtain a timepoint of Day 0 monocytes, then plate remaining cells in 6 well plates in macrophage differentiation medium: DMEM 4.5 ug/ml glucose, Pen-Strep, L-glutamine, 20% FBS and 10% Human AB serum (Gemini, Cat # 100-512). Seed cells at 1.5×10e6 per well (6 well Costar cell culture plates) and grow at 37 C, 7% CO2. After 24 hours in culture, the cells were harvested and lysed in RNA lysis buffer to obtain mRNA for the Day 1 timepoint. The remaining cells were kept in culture and until Day 7. After 7 days in culture, the cells were lysed in RNA lysis buffer to obtain Day 7 timepoint at which time the cells displayed gross macrophage morphology.

The mRNA was isolated by Qiagen miniprep and analysis run on Affimax™ (Affymetrix Inc. Santa Clara, Calif.) microarray chips and proprietary Genentech microarrays. The cells harvested at Day 0 timepoint, the Day 1 timepoint, and the Day 7 timepoint were subjected to the same analysis. Genes were compared whose expression was upregulated at Day 7 as compared to Day 0 and Day 1.

Below are the results of these experiments, demonstrating that various PRO polypeptides of the present invention are differentially expressed in differentiated macrophages at Day 7 as compared to non-differentiated monocytes at Day 0 and at Day 1. As described above, these data demonstrate that the PRO polypeptides of the present invention are useful not only as diagnostic markers for the presence of one or more immune disorders, but also serve as therapeutic targets for the treatment of those immune disorders. Specifically, the cDNAs shown FIG. 592, FIG. 708, FIG. 724, FIG. 888, FIG. 1095, FIG. 1109, FIG. 1456 and FIG. 2331 are significantly overexpressed in differentiated macrophages as compared to non-differentiated monocytes at Day 0 and Day 1.

The FIGS. 1-2517 show the nucleic acids of the invention and their encoded PRO polypeptides that are differentially expressed in differentiated macrophages at Day 7 as compared to non-differentiated monocytes at Day 0 and at Day 1.

### Example 2

### Use of PRO as a Hybridization Probe

The following method describes use of a nucleotide sequence encoding PRO as a hybridization probe.

DNA comprising the coding sequence of full-length or mature PRO as disclosed herein is employed as a probe to screen for homologous DNAs (such as those encoding naturally-occurring variants of PRO) in human tissue cDNA libraries or human tissue genomic libraries.

Hybridization and washing of filters containing either library DNAs is performed under the following high stringency conditions. Hybridization of radiolabeled PRO-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0. 1×SSC and 0.1% SDS at 42° C.

DNAs having a desired sequence identity with the DNA encoding full-length native sequence PRO can then be identified using standard techniques known in the art.

### Example 3

### Expression of PRO in E. coli

This example illustrates preparation of an unglycosylated form of PRO by recombinant expression in E. coli.

The DNA sequence encoding PRO is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al.,. Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO protein can then be purified using a metal chelating column under conditions that allow tight binding of the protein.

PRO may be expressed in E. coli in a poly-His tagged form, using the following procedure. The DNA encoding PRO is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SP in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Many of the PRO polypeptides disclosed herein were successfully expressed as described above.

### Example 4

### Expression of PRO in Mammalian Cells

This example illustrates preparation of a potentially glycosylated form of PRO by recombinant expression in mammalian cells.

The vector, pRK5 (see EP 307,247, published Mar. 15, 1989), is employed as the expression vector. Optionally, the PRO DNA is ligated into pRK5 with selected restriction enzymes to allow insertion of the PRO DNA using ligation methods such as described in Sambrook et al., supra. The resulting vector is called pRK5-PRO.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO DNA is mixed with about 1 μg DNA encoding the VA RNA gene [Thimmappaya et al., Cell, 31:543 (1982)] and dissolved in 500 μL of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/mil ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, PRO may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO can then be concentrated and purified by any selected method, such as dialysis and/or column chromatography.

In another embodiment, PRO can be expressed in CHO cells. The pRK5-PRO can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO polypeptide, the culture medium may be replaced with serum free medium. Preferably, the cultures are incubated for about 6 days, and then the conditioned medium is harvested. The medium containing the expressed PRO can then be concentrated and purified by any selected method.

Epitope-tagged PRO may also be expressed in host CHO cells. The PRO may be subcloned out of the pRK5 vector. The subclone insert can undergo PCR to fuse in frame with a selected-epitope tag such as a poly-his tag into a Baculovirus expression vector. The poly-his tagged PRO insert can then be subcloned into a SV40 promoter/enhancer containing vector containing a selection marker such as DHFR for selection of stable clones. Finally, the CHO cells can be transfected (as described above) with the SV40 promoter/enhancer containing vector. Labeling may be performed, as described above, to verify expression. The culture medium containing the expressed poly-His tagged PRO can then be concentrated and purified by any selected method, such as by Ni²⁺-chelate affinity chromatography.

PRO may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mL of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 5

### Expression of PRO in Yeast

The following method describes recombinant expression of PRO in yeast.

First, yeast expression vectors are constructed for intracellular production or secretion of PRO from the ADH2/GAPDH promoter. DNA encoding PRO and the promoter is inserted into suitable restriction enzyme sites in the selected plasmid to direct intracellular expression of PRO. For secretion, DNA encoding PRO can be cloned into the selected plasmid, together with DNA encoding the ADH2/GAPDH promoter, a native PRO signal peptide or other mammalian signal peptide, or, for example, a yeast alpha-factor or invertase secretory signal/leader sequence, and linker sequences (if needed) for expression of PRO.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO can subsequently be isolated and purified by removing the yeast cells from the fermentation medium by centrifugation and then concentrating the medium using selected cartridge filters. The concentrate containing PRO may further be purified using selected column chromatography resins.

Many of the PRO polypeptides disclosed herein were successfully expressed as described above.

### Example 6

### Expression of PRO in Baculovirus-Infected Insect Cells

The following method describes recombinant expression of PRO in Baculovirus-infected insect cells.

The sequence coding for PRO is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO or the desired portion of the coding sequence of PRO such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., Nature 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fc tagged) PRO can be performed using known chromatography techniques, including for instance, Protein A or protein G column chromatography.

Many of the PRO polypeptides disclosed herein were successfully expressed as described above.

### Example 7

### Preparation of Antibodies that Bind PRO

This example illustrates preparation of monoclonal antibodies which can specifically bind PRO.

Techniques for producing the monoclonal antibodies are known in the art and are described, for instance, in Goding, supra. Immunogens that may be employed include purified PRO, fusion proteins containing PRO, and cells expressing recombinant PRO on the cell surface. Selection of the immunogen can be made by the skilled artisan without undue experimentation.

Mice, such as Balb/c, are immunized with the PRO immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO antibodies.

After a suitable antibody titer has been detected, the animals “positive” for antibodies can be injected with a final intravenous injection of PRO. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO. Determination of “positive” hybridoma cells secreting the desired monoclonal antibodies against PRO is within the skill in the art.

The positive hybridoma cells can be injected intraperitoneally into syngeneic Balb/c mice to produce ascites containing the anti-PRO monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of PRO Polypeptides Using Specific Antibodies

Native or recombinant PRO polypeptides may be purified by a variety of standard techniques in the art of protein purification. For example, pro-PRO polypeptide, mature PRO polypeptide, or pre-PRO polypeptide is purified by immunoaffinity chromatography using antibodies specific for the PRO polypeptide of interest. In general, an immunoaffinity column is constructed by covalently coupling the anti-PRO polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE (Pharmacia LKB Biotechnology). The antibody is coupled to the resin, the resin is blocked, and the derivative resin is washed according to the manufacturer's instructions.

Such an immunoaffinity column is utilized in the purification of PRO polypeptide by preparing a fraction from cells containing PRO polypeptide in a soluble form. This preparation is derived by solubilization of the whole cell or of a subcellular fraction obtained via differential centrifugation by the addition of detergent or by other methods well known in the art. Alternatively, soluble PRO polypeptide containing a signal sequence may be secreted in useful quantity into the medium in which the cells are grown.

A soluble PRO polypeptide-containing preparation is passed over the immunoaffinity column, and the column is washed under conditions that allow the preferential absorbance of PRO polypeptide (e.g., high ionic strength buffers in the presence of detergent). Then, the column is eluted under conditions that disrupt antibody/PRO polypeptide binding (e.g., a low pH buffer such as approximately pH 2-3, or a high concentration of a chaotrope such as urea or thiocyanate ion), and PRO polypeptide is collected.

### Example 9

### Drug Screening

This invention is particularly useful for screening compounds by using PRO polypeptides or binding fragment thereof in any of a variety of drug screening techniques. The PRO polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO polypeptide or fragment. Drugs are screened against such transformed cells in competitive binding assays. Such cells, either in viable or fixed form, can be used for standard binding assays. One may measure, for example, the formation of complexes between PRO polypeptide or a fragment and the agent being tested. Alternatively, one can examine the diminution in complex formation between the PRO polypeptide and its target cell or target receptors caused by the agent being tested.

Thus, the present invention provides methods of screening for drugs or any other agents which can affect a PRO polypeptide-associated disease or disorder. These methods comprise contacting such an agent with an PRO polypeptide or fragment thereof and assaying (I) for the presence of a complex between the agent and the PRO polypeptide or fragment, or (ii) for the presence of a complex between the PRO polypeptide or fragment and the cell, by methods well known in the art. In such competitive binding assays, the PRO polypeptide or fragment is typically labeled. After suitable incubation, free PRO polypeptide or fragment is separated from that present in bound form, and the amount of free or uncomplexed label is a measure of the ability of the particular agent to bind to PRO polypeptide or to interfere with the PRO polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO polypeptide, the peptide test compounds are reacted with PRO polypeptide and washed. Bound PRO polypeptide is detected by methods well known in the art. Purified PRO polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO polypeptide specifically compete with a test compound for binding to PRO polypeptide or fragments thereof. In this manner, the antibodies can be used to detect the presence of any peptide which shares one or more antigenic determinants with PRO polypeptide.

### Example 10

### Rational Drug Design

The goal of rational drug design is to produce structural analogs of biologically active polypeptide of interest (i.e., a PRO polypeptide) or of small molecules with which they interact, e.g., agonists, antagonists, or inhibitors. Any of these examples can be used to fashion drugs which are more active or stable forms of the PRO polypeptide or which enhance or interfere with the function of the PRO polypeptide in vivo (c.f., Hodgson, Bio/Technology. 9: 19-21 (1991)).

In one approach, the three-dimensional structure of the PRO polypeptide, or of a PRO polypeptide-inhibitor complex, is determined by x-ray crystallography, by computer modeling or, most typically, by a combination of the two approaches. Both the shape and charges of the PRO polypeptide must be ascertained to elucidate the structure and to determine active site(s) of the molecule. Less often, useful information regarding the structure of the PRO polypeptide may be gained by modeling based on the structure of homologous proteins. In both cases, relevant structural information is used to design analogous PRO polypeptide-like molecules or to identify efficient inhibitors. Useful examples of rational drug design may include molecules which have improved activity or stability as shown by Braxton and Wells, Biochemistry. 31:7796-7801 (1992) or which act as inhibitors, agonists, or antagonists of native peptides as shown by Athauda et al., J. Biochem. 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO polypeptide may be made available to perform such analytical studies as X-ray crystallography. In addition, knowledge of the PRO polypeptide amino acid sequence provided herein will provide guidance to those employing computer modeling techniques in place of or in addition to x-ray crystallography.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed:

1. Isolated nucleic acid having at least 80% nucleic acid sequence identity to a nucleotide sequence identity to:
(a) the nucleotide sequence shown in any one of the FIGS. 1-2517 (SEQ ID NOS: 1-2517); or
(b) the nucleotide sequence encoding the polypeptide shown in any one of the FIGS. 1-2517 (SEQ ID NOS: 1-2517).

2. A vector comprising the nucleic acid of claim 1.

3. The vector of claim 2 operably linked to control sequences recognized by a host cell transformed with the vector.

4. A host cell comprising the vector of claim 2.

5. The host cell of claim 4, wherein said cell is a CHO cell, an E. coli cell or a yeast cell.

6. A process for producing a PRO polypeptide comprising culturing the host cell of claim 5 under conditions suitable for expression of said PRO polypeptide and recovering said PRO polypeptide from the cell culture.

7. An isolated polypeptide having at least 80% amino acid sequence identity to:
(a) a polypeptide shown in any one of FIGS. 1-2517 (SEQ ID NOS: 1-2517); or
(b) a polypeptide encoded by the full length coding region of the nucleotide sequence shown in any one of FIGS. 1-2517 (SEQ ID NOS: 1-2517).

8. A chimeric molecule comprising a polypeptide according to claim 7 fused to a heterologous amino acid sequence.

9. The chimeric molecule of claim 8, wherein said heterologous amino acid sequence is an epitope tag sequence or an Fc region of an immunoglobulin.

10. An antibody which specifically binds to a polypeptide according to claim 7.

11. The antibody of claim 10, wherein said antibody is a monoclonal antibody, a humanized antibody or a single-chain antibody.

12. A composition of matter comprising (a) a polypeptide of claim 7, (b) an agonist of said polypeptide, (c) an antagonist of said polypeptide, or (d) an antibody that binds to said polypeptide, in combination with a carrier.

13. The composition of matter of claim 12, wherein said carrier is a pharmaceutically acceptable carrier.

14. The composition of matter of claim 13 comprising a therapeutically effective amount of (a), (b), (c) or (d).

15. An article of manufacture, comprising:
a container;
a label on said container; and
a composition of matter comprising (a) a polypeptide of claim 7, (b) an agonist of said polypeptide, (c) an antagonist of said polypeptide, or (d) an antibody that binds to said polypeptide, contained within said container, wherein label on said container indicates that said composition of matter can be used for treating an immune related disease.

16. A method of treating an immune related disorder in a mammal in need thereof comprising administering to said mammal a therapeutically effective amount of (a) a polypeptide of claim 7, (b) an agonist of said polypeptide, (c) an antagonist of said polypeptide, or (d) an antibody that binds to said polypeptide.

17. The method of claim 16, wherein the immune related disorder is systemic lupus erythematosis, rheumatoid arthritis, osteoarthritis, juvenile chronic arthritis, a spondyloarthropathy, systemic sclerosis, an idiopathic inflammatory myopathy, Sjögren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barré syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection or graft-versus-host-disease.

18. A method for determining the presence of a PRO polypeptide of the invention as described in FIGS. 1-2517 (SEQ ID NOS: 1-2517), in a sample suspected of containing said polypeptide, said method comprising exposing said sample to an anti-PRO antibody, where the and determining binding of said antibody to a component of said sample.

19. A method of diagnosing an immune related disease in a mammal, said method comprising detecting the level of expression of a gene encoding a PRO polypeptide of the invention as described in FIGS. 1-2517 (SEQ ID NOS: 1-2517), (a) in a test sample of tissue cells obtained from the mammal, and (b) in a control sample of known normal tissue cells of the same cell type, wherein a higher or lower level of expression of said gene in the test sample as compared to the control sample is indicative of the presence of an immune related disease in the mammal from which the test tissue cells were obtained.

20. A method of diagnosing an immune related disease in a mammal, said method comprising (a) contacting a PRO polypeptide of the invention as described in FIGS. 1-2517 (SEQ ID NOS: 1-2517), anti-PRO antibody with a test sample of tissue cells obtained from said mammal and (b) detecting the formation of a complex between the antibody and the polypeptide in the test sample, wherein formation of said complex is indicative of the presence of an immune related disease in the mammal from which the test tissue cells were obtained.

21. A method of identifying a compound that inhibits the activity of a PRO polypeptide of the invention as described in FIGS. 1-2517 (SEQ ID NOS: 1-2517), said method comprising contacting cells which normally respond to said polypeptide with (a) said polypeptide and (b) a candidate compound, and determining the lack responsiveness by said cell to (a).

22. A method of identifying a compound that inhibits the expression of a gene encoding a PRO polypeptide of the invention as described in FIGS. 1-2517 (SEQ ID NOS: 1-2517), said method comprising contacting cells which normally express said polypeptide with a candidate compound, and determining the lack of expression said gene.

23. The method of claim 22, wherein said candidate compound is an antisense nucleic acid.

24. A method of identifying a compound that mimics the activity of a PRO polypeptide of the invention as described in any one of FIGS. 1-2517 (SEQ ID NOS: 1-2517), said method comprising contacting cells which normally respond to said polypeptide with a candidate compound, and determining the responsiveness by said cell to said candidate compound.

25. A method of stimulating the immune response in a mammal, said method comprising administering to said mammal an effective amount of a PRO polypeptide of the invention as described in any one of FIGS. 1-2517 (SEQ ID NOS: 1-2517), antagonist, wherein said immune response is stimulated.

26. A method of diagnosing an inflammatory immune response in a mammal, said method comprising detecting the level of expression of a gene encoding a PRO polypeptide of the invention as described in any one of FIGS. 1-2517 (SEQ ID NOS: 1-2517), (a) in a test sample of tissue cells obtained from the mammal, and (b) in a control sample of known normal tissue cells of the same cell type, wherein a higher or lower level of expression of said gene in the test sample as compared to the control sample is indicative of the presence of an inflammatory immune response in the mammal from which the test tissue cells were obtained.

27. A method of differentiating monocytes comprising;
(a) isolating a population of monocytes;
(b) contacting the monocytes with an effective amount of a PRO polypeptide of the invention as described in any of of FIGS. 1-2517 (SEQ ID NOS: 1-2517); and
(c) determining the differentiation of said monocytes to said PRO polypeptide.
